(19) United States
(12) Patent Application Publication
(10) Pub. No.: US 2025/0054581 A1
(43) Pub. Date: Feb. 13, 2025

(54) COMPOSITIONS, SYSTEMS, AND METHODS FOR DATA STORAGE BY MODIFYING AND READING CLUSTERS OF CONVERTIBLE MONOMERS IN POLYMERS

(71) Applicant: NAIO, Inc., San Carlos, CA (US)

(72) Inventor: Eric Kool, San Carlos, CA (US)

(21) Appl. No.: 18/809,812

(22) Filed: Aug. 20, 2024

Related U.S. Application Data
(63) Continuation of application No. PCT/US2023/062991, filed on Feb. 22, 2023.
(60) Provisional application No. 63/374,733, filed on Sep. 6, 2022.
(60) Provisional application No. 63/268,354, filed on Feb. 22, 2022.

(51) Int. Cl. G16B 50/30; G06F 3/06

(52) U.S. Cl. CPC G16B 50/30; G06F 3/0604; G06F 3/0655; G06F 3/0673

(57) ABSTRACT

Data encodable polymers, including nucleic polymers, for data storage and related methods are described. Generally, a nucleic acid polymer can have chemically modifiable structures iteratively repeated along the polymer. The chemically modifiable structures can be modified to encode data. A data encoding device can have a spatial resolution that modifies a string of contiguous chemically modifiable structures to yield a cluster of modified structures. Various methods can be utilized to generate a data encodable nucleic acid polymer. Various methods can be utilized to encode a data encodable nucleic acid polymer. Various methods of decoding can be utilized to decode an encoded nucleic acid or other polymer.

### CROSS REFERENCE TO RELATED APPLICATIONS

This application is a continuation application of International Patent Application No. PCT/US2023/062991, filed Feb. 22, 2023, which claims the benefit of U.S. Provisional Patent Applications No. 63/268,354, filed Feb. 22, 2022, and No. 63/374,733 filed Sep. 6, 2022, each of which is hereby incorporated herein by reference in its entirety.

### SEQUENCE LISTING

The instant application contains a Sequence Listing which has been submitted electronically in XML format and is hereby incorporated by reference in its entirety. Said XML copy, created on Feb. 20, 2023, is named NAIO_706.601_SL.xml and is 9,568 bytes in size and 12,288 bytes in size on disk.

### BACKGROUND

The use of polymeric molecules to encode digital data is under highly active research. In principle, a sequence of monomers or iteratively spaced functional groups attached to select monomers can encode ones and zeros (bits) in the binary sense. Such molecular data encoding offers the possibility of very high data density, since a single molecule can encode many bits of data. Moreover, some polymers (such as DNA) can remain stable longer than electronic or magnetic media, thus obviating the need for frequent re-writing of data for archival storage.

Because of the ease of synthesis and analysis of DNA, this biopolymer has received by far the most attention in molecular data encoding studies. Solid-phase synthesis allows assembly of DNA into a desired and/or arbitrary sequence that can encode a string of binary data. In addition, sequencing methods are rapidly becoming efficient and inexpensive, enabling one to “read” the data so encoded. However, the step-by-step assembly of DNA via solid-phase synthesis limits the polymer's length to about 200 monomers, severely limiting the amount of data that can be encoded in a single molecule. Ideally, data would be encoded in very long DNA polymers (for instance, greater than 10 kilobases), but methods for making very long DNAs of arbitrary sequence are limited, slow, or nonexistent. Biologically synthesized DNAs, while very long, do not exist in arbitrary sequence, limiting the ability to encode data efficiently in these polymers.

### SUMMARY

When utilizing single-dye imaging to read and write data stored on biological polymers, single-dye imaging may require highly specialized, sensitive cameras due to the very weak fluorescence emitted by a single chromophore, for example, when considering a single chromophore molecule attached to a single nucleobase of a DNA polymer. Computational analysis may be required to localize the position of the dye in the image relative to other dyes which may be obscured by diffraction. Moreover, where single photomodifiable groups are used as bits, one must resolve one bit from the adjacent one, as a missing one will result in an error in the string of bits. In addition, impinging light on a single dye to modulate it may occur with some stochastic variation, and thus there could be occasional errors comprising not modifying a chromophore when writing was intended. For example, methods may comprise removing a dye from a linker attached to the DNA backbone via light, as to encode digital information, and knowledge and manipulation of a position of a dye's position relative to secondary structures in the DNA may be needed in nanopore sequencers. Thus, where methods comprise nanopore sequencing, forward and reverse current to move the DNA back and forth may be required in order to process information at the single nucleotide level, in addition to highly specialized, sensitive cameras and computational analysis to localize the position of the dye in the image relative to other dyes which may be obscured by diffraction, and errors resulting from stochastic variation, all of which represent a barrier to the adoption of such improved data storage technologies.

Responsive to such barriers within the art, provided herein are methods for the use of chemically modifiable structures are incorporated into a biological polymer, such as DNA or RNA, such as dye clusters in DNA or RNA, to encode bits, wherein the clusters comprise sizes greater than those obscured by diffraction or other optical aberrations. Such embodiments, can allow for the data encoded in the polymer to be read without using single-molecule imaging methods, may enable using standard microscopy cameras or detectors to read the information encoded therein. In some embodiments, provided herein are methods and compositions comprising clusters which may enable writing and reading data at faster times with less expense.

Aspects disclosed herein provide a method of encoding data onto a writable polymer, comprising: providing a data encodable nucleic acid polymer having a sequence of nucleotides in which one or more chemically modifiable structures are iteratively repeated, wherein each chemically modifiable structure of the one or more chemically modifiable structures are attached onto the nucleic acid polymer; and wherein the one or more chemically modifiable structures are capable of being modified into a second structural state by pulses of light energy or of redox energy; and selectively modifying, utilizing a data encoding device, a subset of the one or more chemically modifiable structures along the nucleic acid polymer into the second structural state such that a data encoded polymer is generated as defined by a plurality of modified structures, wherein the modified structures are arranged in clusters of iteratively repeating units, and wherein the length of each cluster is determined by a spatial resolution of a data encoding device. In some embodiments, the clusters comprise at least two consecutively modified structures. In some embodiments, the clusters comprise at least four consecutively modified structures. In some embodiments, the clusters comprise at least 21 consecutively modified structures. In some embodiments, the selectively modifying comprises forming a single cluster at a time. In some embodiments, In some embodiments, the selectively modifying comprises simultaneously modifying a plurality of the chemically modifiable structures to form a cluster. In some embodiments, the selectively modifying comprises modifying a first subset of one or more chemically modifiable structures along the nucleic acid polymer to form a first cluster, not modifying a second subset of one or more chemically modifiable structures positioned along the nucleic acid polymer positioned after the first cluster in a 5′ to 3′ direction, and then modifying a third subset of one or more chemically modifiable structures along the nucleic acid polymer to form a third cluster positioned along the nucleic acid polymer positioned after the second subset of one or more chemically modifiable structures in a 5′ to 3′ direction. In some embodiments, the clusters of repeating units comprise consecutively repeating units. In some embodiments, the clusters of consecutively repeating units comprise at least 2, 3, 4, 5, 6, 7, 8, 9, 10, 12, 16, 20, or 21 repeating units of modified structures. In some embodiments, the clusters of consecutively repeating units comprise at least 21 repeating units. In some embodiments, the clusters of consecutively repeating units comprise at least 625 repeating units. In some embodiments, the clusters of consecutively repeating units comprise at least about 0.6 kbp of repeating units. In some embodiments, the clusters of consecutively repeating units comprise at least about 1.2 kbp of repeating units. In some embodiments, the clusters of consecutively repeating units define a data bit resolution defined by a distance of at least 0.3 microns about the length of the polymer. In some embodiments, the clusters of consecutively repeating units define a data bit resolution defined by a distance of at least 0.6 microns about the length of the polymer. In some embodiments, the clusters of consecutively repeating units define a data bit resolution defined by a distance of at least 1 about microns about the length of the polymer. In some embodiments, the clusters of repeating units are iteratively repeated at least every 4, 5, 6, 7, 8, 9, 10, 12, 16, 20, 21, 24, 28, 32, 40, 80, 120, or 625 nucleotides. In some embodiments, the clusters of repeating units are iteratively repeated at least every 21 units. In some embodiments, the clusters of repeating units are iteratively repeated at least every 625 units. In some embodiments, the clusters of repeating units comprise at least about 0.6 kbp of repeating units. In some embodiments, the clusters of repeating units comprise at least about 1.2 kbp of repeating units. In some embodiments, the clusters of repeating units define a data bit resolution defined by a distance of at least 0.3 microns about the length of the polymer. In some embodiments, the clusters of repeating units define a data bit resolution defined by a distance of at least 0.6 microns about the length of the polymer. In some embodiments, the clusters of repeating units define a data bit resolution defined by a distance of at least 1 about microns about the length of the polymer. In some embodiments, the data encoding device comprise a nanopore, and the method further comprises: passing the encodable nucleic acid polymer through the nanopore of the data encoding device, wherein the nanopore comprises a means to impinge pulses of light energy or redox energy onto the subset of the one or more chemically modifiable structures into the second structural state. In some embodiments, each chemically modifiable structure of the one or more chemically modifiable structures comprises: a caging group, a quencher, a photobleachable fluorophore, a photoconvertible fluorophore, or any combination thereof. In some embodiments, a first chemically modifiable structure of the one or more chemically modifiable structures is a photobleachable fluorophore capable of being photobleached by pulses of light energy at a first wavelength of light. In some embodiments, a second chemically modifiable structure of the one or more chemically modifiable structures is a photobleachable fluorophore capable of being photobleached by pulses of light energy at a second wavelength of light. In some embodiments, the first chemically modifiable structure of the one or more chemically modifiable structures is a photobleachable fluorophore unable of being photobleached by pulses of light energy at the second wavelength of light, and wherein the second chemically modifiable structure of the one or more chemically modifiable structures is a photobleachable fluorophore unable of being photobleached by pulses of light energy at the first wavelength of light. In some embodiments, the first and the second chemically modifiable structures are attached to a repeated nucleotide structure. In some embodiments, the nucleic acid polymer is double stranded, wherein the first chemically modifiable structure is attached to a repeated nucleotide structure on a top strand of the nucleic acid polymer and the second chemically modifiable structure is attached to a repeated nucleotide structure on a bottom strand of the nucleic acid polymer. In some embodiments, the one or more chemically modifiable structures are iteratively repeated as follows: every other nucleotide, every 3rd nucleotide, every 4th nucleotide, every 5th nucleotide, every 6th nucleotide, every 7th nucleotide, every 8th nucleotide, every 9th nucleotide, every 10th nucleotide, every 11th nucleotide, every 12th nucleotide, every 13th nucleotide, every 14th nucleotide, every 15th nucleotide, every 16th nucleotide, every 17th nucleotide, every 18th nucleotide, every 19th nucleotide, every 20th nucleotide, every 21st nucleotide, every 22nd nucleotide, every 23rd nucleotide, every 24th nucleotide, or every 25th nucleotide. In some embodiments, the polymer comprises DNA, RNA, phosphorothioate DNA, glycerol nucleic acids (GNA), threose nucleic acids (TNA), 2′-fluoro-DNA 2′-O-methyl RNA, or locked nucleic acids (LNA). In some embodiments, the method further includes: decoding the encoded data of the selectively modified nucleic acid polymer. In some embodiments, the encoded data is decoded by passing the selectively modified nucleic acid polymer through a nanopore of a data decoding device, wherein the nanopore of the data decoding device comprises a means to detect the chemically modified structures within each cluster. In some embodiments, the data decoding device is the same device as the data encoding device. In some embodiments, the nanopore to decode data is the same nanopore to encode data. In some embodiments, the encoded data is decoded by stretching and imaging the selectively modified nucleic acid polymer. In some embodiments, the photo-modifiable group comprises a photo-removable leaving group not attached by a linker. In some embodiments, the photo-modifiable group comprises a photo-removable leaving group cleavable at a carbonyl bond, a thio (S) bond, or a NR2 bond, by light. In some embodiments, the plurality of convertible nucleobases are converted from the first state into the second state by light of a wavelength of 325 nm, 360 nm, or 400 nm. In some embodiments, the plurality of convertible nucleobases are converted from the first state into the second state by light of a wavelength of between 400 nm to 850 nm. In some embodiments, at least three convertible residues in the first state are between data encoding segments of the polymer which comprise a plurality of convertible residues in the second state. In some embodiments, at least three convertible residues in the first state are utilized for each nanometer of a spatial resolution in which the data is encoded. In some embodiments the method further includes a plurality of spacers in between data encoding segments of the polymer. In some embodiments, at least three spacer residues are utilized for each nanometer of a spatial resolution in which the data is encoded. In some embodiments, spacer residues are nucleobases which are not photo-modifiable. In some embodiments, the convertible residues comprise covalently attached monomer units. In some embodiments, the convertible wherein the chemically modifiable structures comprise a photo-modifiable group comprising a photo-removable leaving group. In some embodiments, the convertible residues comprise covalently attached monomer units which are continuously linked about the entire length of the polymer. In some embodiments, the convertible residues comprise covalently attached monomer units, wherein the data is encoded in the covalently linked monomer units. In some embodiments, the convertible residues comprise covalently attached monomer units, wherein the data is encoded in continuously covalently linked monomer units. In some embodiments, the polymer is defined by covalently linked monomer units, wherein the polymer is not hybridized in a side by side direction. In some embodiments, the polymer is defined by covalently linked monomer units, wherein the polymer is not hybridized in a side by side direction to a second polymer to which the polymer is not covalently linked. In some embodiments the method further includes forming a single stranded polymer. In some embodiments, the writable polymer is DNA, further comprising producing a second polymer which is complementary to the data encoded polymer at about at least a portion of the data encoded polymer. In some embodiments the method further includes hybridizing at least a portion of the data encoded polymer to the second polymer. In some embodiments the method further includes hybridizing at least a portion of the data encoded polymer to the second polymer in a 5′ to 3′ direction. In some embodiments, the polymer comprises up to two continuous covalently linked polymer molecules. In some embodiments, the two continuous covalently linked polymer molecules form a DNA duplex. In some embodiments, the DNA duplex is hybridized in a 3′ to 5′ direction. In some embodiments, the iteratively spaced convertible residues are positioned on a single covalently linked polymer. In some embodiments the method further includes heating, cooling, reheating, or any combination thereof the data encoded polymer, wherein the data encoded polymer maintains the position of its residues about a 3′ to 5′ direction during the heating, cooling, reheating, or any combination thereof. In some embodiments, the chemically modifiable moiety is directly linked to one or more nucleobases. In some embodiments, the chemically modifiable moiety is iteratively spaced along about the length of the polymer. The method of claim 58, wherein the iterative spacing along the length of the polymer occurs by at least every 2, 3, 4, 5, 6, 7, 8, 9, 10, 20, 30, 40, 50, 100, 500, 1000, 2500, or 5000 spacer residues. In some embodiments, the selectively modifying comprises removing a photo-removable leaving group at least every 2, 3, 4, 5, 6, 7, 8, 9, 10, 20, 30, 40, 50, 100, 500, 1000, 2500, or 5000 spacer residues. In some embodiments, the selectively modifying comprises locally photocleaving a single photo-removable leaving group at a time. In some embodiments, there are no secondary structures attached to the polymer except for the chemically modifiable moiety directly linked to the one or more nucleobases. In some embodiments, the polymer does not encode any data when the plurality of convertible residues are in the first state. In some embodiments, the polymer is a writable polymer configured for the writing of data onto the polymer. In some embodiments, the polymer encodes data when at least a portion of the plurality of convertible residues are in the second state. In some embodiments, the selectively modifying encodes data by cleaving at least a portion of the photo-removable leaving groups from the chemically modifiable moiety to form the data encoded polymer. In some embodiments, the selectively modifying comprises cleaving the photo-removable leaving group from a nucleobase of the one or more nucleobases without using a reagent. In some embodiments, selectively modifying comprises moving the polymer through a nanopore. In some embodiments, the selectively modifying comprises moving the polymer through a nanopore in a unidirectional manner without moving the polymer through the nanopore in a reverse direction. In some embodiments, the selectively modifying comprises moving the polymer through a nanopore at a continuous speed. In some embodiments, the selectively modifying comprises writing data along the length of the polymer for a length of at least 1000, 2000, 3000, 4000, 5000, 10000, or 500000 residues. In some embodiments, the selectively modifying does not comprise oxidizing the one or more nucleobases. In some embodiments, the chemically modifiable moiety comprises a molecule comprising a plurality of distinct atoms. In some embodiments, the chemically modifiable moiety comprises a molecule comprising atoms other than Oxygen (O). In some embodiments, the photoremovable leaving group comprises a molecule comprising a plurality of atoms. In some embodiments, the photoremovable leaving group comprises a molecule comprising a plurality of distinct atoms. In some embodiments, the photoremovable leaving group comprises a molecule comprising atoms other than O. In some embodiments the method further includes passing the writable polymer encoded with data through a data reading device to read the encoded data on the polymer by identifying whether each of the plurality of convertible residues passing through the data reading device comprises the photo-removable leaving group.

Aspects disclosed herein provide a polymer encoded with data utilizing a data encoding with a resolution to yield iteratively spaced clusters of modified structures, comprising: a nucleic acid polymer having a sequence of nucleotides in which one or more chemically modifiable structures are iteratively repeated; wherein the nucleic acid polymer comprises clusters of repeating units of modified structures, where the clusters are iteratively repeated along the nucleic acid; wherein the modified structures have been chemically modified from a first structural state into a second structural state via pulses of light or redox energy; and wherein the plurality of clusters define encoded data as determined by the modified structures within each cluster. In some embodiments, the clusters of repeating units comprise consecutively repeating units of the modified structures in the second structural state. In some embodiments, the clusters of consecutively repeating units comprise at least 2, 3, 4, 5, 6, 7, 8, 9, or 10 repeating units of modified structures. In some embodiments, the clusters of consecutively repeating units comprise at least 21 repeating units of modified structures. In some embodiments, the clusters of consecutively repeating units comprise at least 625 repeating units of modified structures. In some embodiments, the clusters of consecutively repeating units comprise at least about 0.6 kbp of repeating units of modified structures. In some embodiments, the clusters of consecutively repeating units comprise at least about 1.2 kbp of repeating units of modified structures. In some embodiments, the clusters of consecutively repeating units define a data bit resolution defined by a distance of at least 0.3 microns about the length of the polymer. In some embodiments, the clusters of consecutively repeating units define a data bit resolution defined by a distance of at least 0.6 microns about the length of the polymer. In some embodiments, the clusters of consecutively repeating units define a data bit resolution defined by a distance of at least 1 about microns about the length of the polymer. In some embodiments, the clusters of repeating units are iteratively repeated at least every 2, 3, 4, 5, 6, 7, 8, 9, or 10 units. In some embodiments, the clusters of repeating units are iteratively repeated at least every 21 units. In some embodiments, the clusters of repeating units are iteratively repeated at least every 625 units. In some embodiments, the clusters of repeating units comprise at least about 0.6 kbp of repeating units. In some embodiments, the clusters of repeating units comprise at least about 1.2 kbp of repeating units. In some embodiments, the clusters of repeating units define a data bit resolution defined by a distance of at least 0.3 microns about the length of the polymer. In some embodiments, the clusters of repeating units define a data bit resolution defined by a distance of at least 0.6 microns about the length of the polymer. In some embodiments, the clusters of repeating units define a data bit resolution defined by a distance of at least 1 about microns about the length of the polymer. In some embodiments, the chemically modifiable structures comprise a photo-modifiable group comprising a photo-removable leaving group. In some embodiments, the photo-modifiable group comprises a photo-removable leaving group not attached by a linker. In some embodiments, the photo-modifiable group comprises a photo-removable leaving group cleavable at a carbonyl bond, a thio (S) bond, or a NR2 bond, by light. In some embodiments, the plurality of convertible nucleobases are converted from the first state into the second state by light of a wavelength of 325 nm, 360 nm, or 400 nm. In some embodiments, the plurality of convertible nucleobases are converted from the first state into the second state by light of a wavelength of between 400 nm to 850 nm. In some embodiments, at least three convertible residues in the first state are between data encoding segments of the polymer which comprise a plurality of convertible residues in the second state. In some embodiments, at least three convertible residues in the first state are utilized for each nanometer of a spatial resolution in which the data is encoded. In some embodiments the polymer further includes a plurality of spacers in between data encoding segments of the polymer. In some embodiments, at least three spacer residues are utilized for each nanometer of a spatial resolution in which the data is encoded. In some embodiments, spacer residues are nucleobases which are not photo-modifiable. In some embodiments, the convertible residues comprise covalently attached monomer units. In some embodiments, the convertible residues comprise covalently linked monomer units which are continuously linked about the entire length of the polymer. In some embodiments, the convertible residues comprise covalently linked monomer units, wherein the data is encoded in the covalently linked monomer units. In some embodiments, the convertible residues comprise covalently linked monomer units, wherein the data is encoded in continuously covalently linked monomer units. In some embodiments, the polymer is defined by covalently linked monomer units, wherein the polymer is not hybridized in a side by side direction. In some embodiments, the polymer is defined by covalently linked monomer units, wherein the polymer is not hybridized in a side by side direction to a second polymer to which the polymer is not covalently linked. In some embodiments, the polymer comprises a single molecule forming a single stranded polymer. In some embodiments, the polymer is defined by up to 2 DNA polymer molecules. In some embodiments, the polymer is defined by up to 2 hybridized DNA polymer molecules. In some embodiments, the polymer is defined by up to 2 hybridized DNA polymer molecules hybridized in a 5′ to 3′ direction. In some embodiments, the polymer comprises up to two continuous covalently linked polymer molecules. In some embodiments, the two continuous covalently linked polymer molecules form a DNA duplex. In some embodiments, the DNA duplex is hybridized in a 3′ to 5′ direction. In some embodiments, the iteratively spaced convertible residues are positioned on a single covalently linked polymer. In some embodiments, the polymer maintains the position of its residues about a 3′ to 5′ direction when heated, cooled, reheated, or any combination thereof. In some embodiments, the polymer is not attached to any other polymer by hybridization. In some embodiments, the polymer comprises one or more nucleobases, wherein the chemically modifiable moiety is directly linked to the one or more nucleobases. In some embodiments, the convertible residues comprising the chemically modifiable moiety are iteratively spaced along about the length of the polymer. In some embodiments, the iterative spacing along the length of the polymer occurs by at least every 2, 3, 4, 5, 6, 7, 8, 9, 10, 20, 30, 40, 50, 100, 500, 1000, 2500, or 5000 spacer residues. In some embodiments, the polymer is configured for local photocleavage of a single removable leaving group at a time. In some embodiments, there are no secondary structures attached to the polymer except for the chemically modifiable moiety directly linked to the one or more nucleobases. In some embodiments, the polymer does not encode any data when the plurality of convertible residues are in the first state. In some embodiments, polymer is a writable polymer configured for the writing of data onto the polymer. In some embodiments, the polymer encode data when at least a portion of the plurality of convertible residues are in the second state. In some embodiments, the polymer is configured to encode data by cleaving at least a portion of the photo-removable leaving groups from the polymer. In some embodiments, the chemically modifiable moiety is configured to be cleaved by the one or more nucleobases without using a chemical reagent. In some embodiments, the polymer is configured to move through a nanopore in a unidirectional manner. In some embodiments, the polymer is configured to move through a nanopore in a unidirectional manner without moving the polymer through the nanopore in a reverse direction. In some embodiments, the polymer is at least 1000, 2000, 3000, 4000, 5000, 10000, or 500000 residues in length. In some embodiments, the polymer in the second state does not comprise an oxidized nucleobase. In some embodiments, the second state is a written form which does not comprise an oxidized nucleobase. In some embodiments, the chemically modifiable moiety comprises a chemical residue comprising a plurality of atoms. In some embodiments, the polymer in the second state does not comprise the chemically modifiable moiety. In some embodiments, the polymer in the second state does not comprise the chemical residue comprising a plurality of atoms. In some embodiments, the chemically modifiable moiety comprises a molecule comprising atoms other than O (oxygen). In some embodiments, the photo-removable leaving group comprises a molecule comprising a plurality of atoms. In some embodiments, the photo-removable leaving group comprises a molecule comprising a plurality of distinct atoms. In some embodiments, the photo-removable leaving group comprises a molecule comprising atoms other than O. In some embodiments, the first structural state of each modified structure of the plurality of modified structures comprises one of the following: a caging group, a quencher, a photobleachable fluorophore, or a photoconvertible fluorophore. In some embodiments, the modified structures of a first cluster are one or more photobleached fluorophores that have been photobleached by pulses of light energy at a first wavelength of light. In some embodiments, the modified structures of a second cluster are one or more photobleached fluorophores that have been photobleached by pulses of light energy at a second wavelength of light. In some embodiments, the first cluster further comprises one or more photobleachable fluorophores capable of being photobleached at the second wavelength of light but have not been photobleached; and wherein the second cluster further comprises one or more photobleachable fluorophores capable of being photobleached at the first wavelength of light but have not been photobleached. In some embodiments, each photobleachable fluorophore capable of being photobleached by the first wavelength within the first cluster has been photobleached by pulses of light energy at the first wavelength of light; and wherein each photobleachable fluorophore capable of being photobleached by the second wavelength within the second cluster has been photobleached by pulses of light energy at the second wavelength of light. In some embodiments, the modified structures and the one or more photobleachable fluorophores of the first cluster are attached to a repeated nucleotide structure. In some embodiments, the nucleic acid is double stranded; wherein the modified structures of the first cluster are attached to a repeated nucleotide structure on a top strand of the nucleic acid polymer and the one or more photobleachable fluorophores of the first cluster are attached to a repeated nucleotide structure on a bottom strand of the nucleic acid polymer. In some embodiments, the length of each cluster of the plurality of clusters is equivalent to a spatial resolution of the pulses of light or redox energy that was used to modify the plurality of chemically modified structures from the first structural state into the second structural state. In some embodiments, the nucleic acid polymer comprises a plurality of spaces between each cluster of the plurality of clusters. In some embodiments, each space of the plurality of spaces comprises one or more chemically modifiable structures that have not been modified by pulses of light or redox energy. In some embodiments, the one or more modified structures are iteratively repeated as follows: every other nucleotide, every 3rd nucleotide, every 4th nucleotide, every 5th nucleotide, every 6th nucleotide, every 7th nucleotide, every 8th nucleotide, every 9th nucleotide, every 10th nucleotide, every 11th nucleotide, every 12th nucleotide, every 13th nucleotide, every 14th nucleotide, every 15th nucleotide, every 16th nucleotide, every 17th nucleotide, every 18th nucleotide, every 19th nucleotide, every 20th nucleotide, every 21st nucleotide, every 22nd nucleotide, every 23rd nucleotide, every 24th nucleotide, or every 25th nucleotide. In some embodiments, the polymer comprises DNA, RNA, phosphorothioate DNA, glycerol nucleic acids (GNA), threose nucleic acids (TNA), 2′-fluoro-DNA 2′-O-methyl RNA, or locked nucleic acids (LNA).

Aspects disclosed herein provide a data encodable polymer for data encoding utilizing iteratively spaced chemically modifiable structures, comprising: a nucleic acid polymer having a sequence of nucleotides in which one or more chemically modifiable structures are iteratively repeated, wherein each chemically modifiable structure of the one or more chemically modifiable structures are attached onto the nucleic acid polymer; and wherein the one or more chemically modifiable structures are capable of being modified by pulses of light energy or of redox energy from a first structural state into a second structural state in clusters of a plurality chemically modified structures into the second structural state. In some embodiments, the clusters of repeating units comprise consecutively repeating units. In some embodiments, the clusters of consecutively repeating units comprise at least 4, 8, 10, 12, 16, or 20 repeating units. In some embodiments, the clusters of consecutively repeating units comprise at least 21 repeating units. In some embodiments, the clusters of consecutively repeating units comprise at least 625 repeating units. In some embodiments, the clusters of consecutively repeating units comprise at least about 0.6 kbp of repeating units. In some embodiments, the clusters of consecutively repeating units comprise at least about 1.2 kbp of repeating units. In some embodiments, the clusters of consecutively repeating units define a data bit resolution defined by a distance of at least 0.3 microns about the length of the polymer. In some embodiments, the clusters of consecutively repeating units define a data bit resolution defined by a distance of at least 0.6 microns about the length of the polymer. In some embodiments, the clusters of consecutively repeating units define a data bit resolution defined by a distance of at least 1 about microns about the length of the polymer. In some embodiments, the clusters of repeating units are iteratively repeated at least every 10 units. In some embodiments, the clusters of repeating units are iteratively repeated at least every 21 units. In some embodiments, the clusters of repeating units are iteratively repeated at least every 625 units. In some embodiments, the clusters of repeating units comprise at least about 0.6 kbp of repeating units. In some embodiments, the clusters of repeating units comprise at least about 1.2 kbp of repeating units. In some embodiments, the clusters of repeating units define a data bit resolution defined by a distance of at least 0.3 microns about the length of the polymer. In some embodiments, the clusters of repeating units define a data bit resolution defined by a distance of at least 0.6 microns about the length of the polymer. In some embodiments, the clusters of repeating units define a data bit resolution defined by a distance of at least 1 about microns about the length of the polymer. In some embodiments, each chemically modifiable structure of the one or more chemically modifiable structures comprises one of the following: a caging group, a quencher, a photobleachable fluorophore, or a photoconvertible fluorophore. In some embodiments, a first chemically modifiable structure of the one or more chemically modifiable structures is a photobleachable fluorophore capable of being photobleached by pulses of light energy at a first wavelength of light. In some embodiments, the first chemically modifiable structure is attached to a repeated nucleotide structure. In some embodiments, a second chemically modifiable structure of the one or more chemically modifiable structures is a photobleachable fluorophore capable of being photobleached by pulses of light energy at a second wavelength of light. In some embodiments, the first chemically modifiable structure of the one or more chemically modifiable structures is a photobleachable fluorophore unable of being photobleached by pulses of light energy at the second wavelength of light, and wherein the second chemically modifiable structure of the one or more chemically modifiable structures is a photobleachable fluorophore unable of being photobleached by pulses of light energy at the first wavelength of light. In some embodiments, the first and the second chemically modifiable structures are attached to a repeated nucleotide structure. In some embodiments, the nucleic acid polymer is double stranded, wherein the first chemically modifiable structure is attached to a repeated nucleotide structure on a top strand of the nucleic acid polymer and the second chemically modifiable structure is attached to a repeated nucleotide structure on a bottom strand of the nucleic acid polymer. In some embodiments, the one or more chemically modifiable structures comprises a photoconvertible fluorophore, wherein the photoconvertible fluorophore exists in a first structural state having a first emission wavelength and is capable of being converted into a second structural state having a second emission wavelength via the pulses of light energy or of redox energy. In some embodiments, the conversion of the photoconvertible fluorophore from the first structural state into a second structural state is via the pulses of light at first wavelength; and wherein the photoconvertible fluorophore is capable of being further converted into a third structural state having a third emission wavelength via the pulses of light at a second wavelength. In some embodiments, the one or more chemically modifiable structures are iteratively repeated as follows: every other nucleotide, every 3rd nucleotide, every 4th nucleotide, every 5th nucleotide, every 6th nucleotide, every 7th nucleotide, every 8th nucleotide, every 9th nucleotide, every 10th nucleotide, every 11th nucleotide, every 12th nucleotide, every 13th nucleotide, every 14th nucleotide, every 15th nucleotide, every 16th nucleotide, every 17th nucleotide, every 18th nucleotide, every 19th nucleotide, every 20th nucleotide, every 21st nucleotide, every 22nd nucleotide, every 23rd nucleotide, every 24th nucleotide, or every 25th nucleotide. In some embodiments, the polymer comprises DNA, RNA, phosphorothioate DNA, glycerol nucleic acids (GNA), threose nucleic acids (TNA), 2′-fluoro-DNA 2′-O-methyl RNA, or locked nucleic acids (LNA). In some embodiments, the chemically modifiable structures comprise a photo-modifiable group comprising a photo-removable leaving group. In some embodiments, the photo-modifiable group comprises a photo-removable leaving group not attached by a linker. In some embodiments, the photo-modifiable group comprises a photo-removable leaving group cleavable at a carbonyl bond, a thio (S) bond, or a NR2 bond, by light. In some embodiments, the plurality of convertible nucleobases are converted from the first state into the second state by light of a wavelength of 325 nm, 360 nm, or 400 nm. In some embodiments, the plurality of convertible nucleobases are converted from the first state into the second state by light of a wavelength of between 400 nm to 850 nm. In some embodiments, at least three convertible residues in the first state are between data encoding segments of the polymer which comprise a plurality of convertible residues in the second state. In some embodiments, at least three convertible residues in the first state are utilized for each nanometer of a spatial resolution in which the data is encoded. In some embodiments the polymer further includes a plurality of spacers in between data encoding segments of the polymer. In some embodiments, at least three spacer residues are utilized for each nanometer of a spatial resolution in which the data is encoded. In some embodiments, spacer residues are nucleobases which are not photo-modifiable. In some embodiments, the convertible residues comprise covalently attached monomer units. In some embodiments, the convertible residues comprise covalently linked monomer units which are continuously linked about the entire length of the polymer. In some embodiments, the convertible residues comprise covalently linked monomer units, wherein the data is encoded in the covalently linked monomer units. In some embodiments, the convertible residues comprise covalently linked monomer units, wherein the data is encoded in continuously covalently linked monomer units. In some embodiments, the polymer is defined by covalently linked monomer units, wherein the polymer is not hybridized in a side by side direction. In some embodiments, the polymer is defined by covalently linked monomer units, wherein the polymer is not hybridized in a side by side direction to a second polymer to which the polymer is not covalently linked. In some embodiments, the polymer comprises a single molecule forming a single stranded polymer. In some embodiments, the polymer is defined by up to 2 DNA polymer molecules. In some embodiments, the polymer is defined by up to 2 hybridized DNA polymer molecules. In some embodiments, the polymer is defined by up to 2 hybridized DNA polymer molecules hybridized in a 5′ to 3′ direction. In some embodiments, the polymer comprises up to two continuous covalently linked polymer molecules. In some embodiments, the two continuous covalently linked polymer molecules form a DNA duplex. In some embodiments, the DNA duplex is hybridized in a 3′ to 5′ direction. In some embodiments, the iteratively spaced convertible residues are positioned on a single covalently linked polymer. In some embodiments, the polymer maintains the position of its residues about a 3′ to 5′ direction when heated, cooled, reheated, or any combination thereof. In some embodiments, the polymer is not attached to any other polymer by hybridization. In some embodiments, the polymer comprises one or more nucleobases, wherein the chemically modifiable moiety is directly linked to the one or more nucleobases. In some embodiments, the convertible residues comprising the chemically modifiable moiety are iteratively spaced along about the length of the polymer. In some embodiments, the iterative spacing along the length of the polymer occurs by at least every 2, 3, 4, 5, 6, 7, 8, 9, 10, 20, 30, 40, 50, 100, 500, 1000, 2500, or 5000 spacer residues. In some embodiments, the polymer is configured for local photocleavage of a single removable leaving group at a time. In some embodiments, there are no secondary structures attached to the polymer except for the chemically modifiable moiety directly linked to the one or more nucleobases. In some embodiments, the polymer does not encode any data when the plurality of convertible residues are in the first state. In some embodiments, polymer is a writable polymer configured for the writing of data onto the polymer. In some embodiments, the polymer encode data when at least a portion of the plurality of convertible residues are in the second state. In some embodiments, the polymer is configured to encode data by cleaving at least a portion of the photo-removable leaving groups from the polymer. In some embodiments, the chemically modifiable moiety is configured to be cleaved by the one or more nucleobases without using a chemical reagent. In some embodiments, the polymer is configured to move through a nanopore in a unidirectional manner. In some embodiments, the polymer is configured to move through a nanopore in a unidirectional manner without moving the polymer through the nanopore in a reverse direction. In some embodiments, the polymer is at least 1000, 2000, 3000, 4000, 5000, 10000, or 500000 residues in length. In some embodiments, the polymer in the second state does not comprise an oxidized nucleobase. In some embodiments, the second state is a written form which does not comprise an oxidized nucleobase. In some embodiments, the chemically modifiable moiety comprises a chemical residue comprising a plurality of atoms. In some embodiments, the polymer in the second state does not comprise the chemically modifiable moiety. In some embodiments, the polymer in the second state does not comprise the chemical residue comprising a plurality of atoms. In some embodiments, the chemically modifiable moiety comprises a molecule comprising atoms other than O (oxygen). The polymer of claim 193, wherein the photo-removable leaving group comprises a molecule comprising a plurality of atoms. In some embodiments, the photo-removable leaving group comprises a molecule comprising a plurality of distinct atoms. In some embodiments, the photo-removable leaving group comprises a molecule comprising atoms other than O In some embodiments, the polymer is configured to encode data in clusters of chemically modified structures converted from the chemically modifiable structures. A data encodable polymer for data encoding utilizing iteratively spaced chemically modifiable structures, comprising: a polymer having a sequence of monomers in which one or more chemically modifiable structures are iteratively repeated in clusters of repeating units, wherein each chemically modifiable structure of the one or more chemically modifiable structures are attached onto the nucleic acid polymer via a monomer; and wherein the one or more chemically modifiable structures are capable of being modified by pulses of light energy or of redox energy. In some embodiments, the clusters of repeating units comprise consecutively repeating units. In some embodiments, the clusters of consecutively repeating units comprise at least 10 repeating units. In some embodiments, the clusters of consecutively repeating units comprise at least 21 repeating units. In some embodiments, the clusters of consecutively repeating units comprise at least 625 repeating units. In some embodiments, the clusters of consecutively repeating units comprise at least about 0.6 kbp of repeating units. In some embodiments, the clusters of consecutively repeating units comprise at least about 1.2 kbp of repeating units. In some embodiments, the clusters of consecutively repeating units define a data bit resolution defined by a distance of at least 0.3 microns about the length of the polymer. In some embodiments, the clusters of consecutively repeating units define a data bit resolution defined by a distance of at least 0.6 microns about the length of the polymer. In some embodiments, the clusters of consecutively repeating units define a data bit resolution defined by a distance of at least 1 about microns about the length of the polymer. In some embodiments, the clusters of repeating units are iteratively repeated at least every 10 units. In some embodiments, the clusters of repeating units are iteratively repeated at least every 21 units. In some embodiments, the clusters of repeating units are iteratively repeated at least every 625 units. In some embodiments, the clusters of repeating units comprise at least about 0.6 kbp of repeating units. In some embodiments, the clusters of repeating units comprise at least about 1.2 kbp of repeating units. In some embodiments, the clusters of repeating units define a data bit resolution defined by a distance of at least 0.3 microns about the length of the polymer. In some embodiments, the clusters of repeating units define a data bit resolution defined by a distance of at least 0.6 microns about the length of the polymer. In some embodiments, the clusters of repeating units define a data bit resolution defined by a distance of at least 1 about microns about the length of the polymer. In some embodiments, each chemically modifiable structure of the one or more chemically modifiable structures comprises one of the following: a caging group, a quencher, a photobleachable fluorophore, or a photoconvertible fluorophore. In some embodiments, a first chemically modifiable structure of the one or more chemically modifiable structures is a photobleachable fluorophore capable of being photobleached by pulses of light energy at a first wavelength of light. In some embodiments, a second chemically modifiable structure of the one or more chemically modifiable structures is a photobleachable fluorophore capable of being photobleached by pulses of light energy at a second wavelength of light. In some embodiments, the first chemically modifiable structure of the one or more chemically modifiable structures is a photobleachable fluorophore unable of being photobleached by pulses of light energy at the second wavelength of light, and wherein the second chemically modifiable structure of the one or more chemically modifiable structures is a photobleachable fluorophore unable of being photobleached by pulses of light energy at the first wavelength of light. In some embodiments, the one or more chemically modifiable structures comprises a photoconvertible fluorophore, wherein the photoconvertible fluorophore exists in a first structural state having a first emission wavelength and is capable of being converted into a second structural state having a second emission wavelength via the pulses of light energy or of redox energy. In some embodiments, the conversion of the photoconvertible fluorophore from the first structural state into a second structural state is via the pulses of light at first wavelength; and wherein the photoconvertible fluorophore is capable of being further converted into a third structural state having a third emission wavelength via the pulses of light at a second wavelength. In some embodiments, the one or more chemically modifiable structures are iteratively repeated as follows: every other monomer, every 3rd monomer, every 4th monomer, every 5th monomer, every 6th monomer, every 7th monomer, every 8th monomer, every 9th monomer, every 10th monomer, every 11th monomer, every 12th monomer, every 13th monomer, every 14th monomer, every 15th monomer, every 16th monomer, every 17th monomer, every 18th monomer, every 19th monomer, every 20th monomer, every 21st monomer, every 22nd monomer, every 23rd monomer, every 24th monomer, or every 25th monomer. In some embodiments, the polymer is an inorganic polymer.

### BRIEF DESCRIPTION OF THE DRAWINGS

The description and claims will be more fully understood with reference to the following figures and data graphs, which are presented as exemplary embodiments and should not be construed as a complete recitation of the scope of the disclosure.

FIG. 1 provides structure drawings of examples of chemically modifiable structures that can be incorporated into nucleic acid polymers in accordance with various embodiments.

FIGS. 2A-2C provide molecular structure diagrams of exemplary sets of chemically modifiable structures for use in a writable polymer in accordance with various embodiments.

FIGS. 3A-3C provide schematic drawings of nucleic acids with iteratively repeated chemically modifiable structures along the polymer in accordance with various embodiments.

FIG. 4 provides diagrams of exemplary reactions to install chemically modifiable structures onto nucleic acid polymers in accordance with various embodiments.

FIG. 5 provides examples of functional groups that can be utilized to install chemically modifiable structures onto polymers in accordance with various embodiments.

FIG. 6 provides a schematic of an exemplary method to encode data by modifying chemically modifiable structures in clusters along a nucleic acid polymer in accordance with various embodiments.

FIG. 7 shows an example of labeled DNA electrophoresis results where Lane 1 contains Cy5-labeled DNA and Lane M contains Safegreen 1 kb DNA ladder in accordance with various embodiments.

FIGS. 8A-8C provide DNA imaging results of Cy-5-labelled DNAs before and after bleaching in accordance with various embodiments. FIG. 8A shows an image of Cy5 labeled DNA before bleaching and FIG. 8B shows an image of Cy5 labeled DNA after localized photobleaching. FIG. 8C shows a plot displaying two cross section profiles: before bleaching (1501) and after bleaching (1502).

FIGS. 9A-9D provides an example of data encoding via photobleaching of clusters of fluorophores on DNA polymers in accordance with various embodiments.

FIGS. 10A-10C provides an examples of data encoded on DNA polymers comprising 42 fluorophores per section in accordance with various embodiments.

FIGS. 11A-11C provides an examples of data encoded on DNA polymers comprising 21 fluorophores per section in accordance with various embodiments.

### DETAILED DESCRIPTION

Various embodiments are directed to compositions and systems of polymeric data storage, methods of use thereof and of synthesis thereof. In several embodiments, writable polymers are generated by generating polymers with iterated repeating chemically modifiable structures. In many embodiments, chemically modifiable structures are incorporated into a biological polymer, such as DNA or RNA. In several embodiments, data is encoded into a polymer by modifying the chemically modifiable structures in clusters. In many embodiments, the length of a cluster is defined by the resolution provided by the writing device or method. Modification of structures comprises uncaging fluorophores, releasing of quenchers, uncaging DNA bases, and/or photoconverting or photobleaching fluorescent dyes, which can be done chemically by utilizing light or redox energy.

When utilizing single-dye imaging to read and write data stored on biological polymers, single-dye imaging may require highly specialized, sensitive cameras due to the very weak fluorescence emitted by a single chromophore, for example, when considering a single chromophore molecule attached to a single nucleobase of a DNA polymer. Computational analysis may be required to localize the position of the dye in the image relative to other dyes which may be obscured by diffraction. Moreover, where single photomodifiable groups are used as bits, one must resolve one bit from the adjacent one, as a missing one will result in an error in the string of bits. In addition, impinging light on a single dye to modulate it may occur with some stochastic variation, and thus there could be occasional errors comprising not modifying a chromophore when writing was intended. For example, methods may comprise removing a dye from a linker attached to the DNA backbone via light, as to encode digital information, and knowledge and manipulation of a position of a dye's position relative to secondary structures in the DNA may be needed in nanopore sequencers. Thus, where methods comprise nanopore sequencing, forward and reverse current to move the DNA back and forth may be required in order to process information at the single nucleotide level, in addition to highly specialized, sensitive cameras and computational analysis to localize the position of the dye in the image relative to other dyes which may be obscured by diffraction, and errors resulting from stochastic variation, all of which represent a barrier to the adoption of such improved data storage technologies.

Responsive to such barriers within the art, provided herein are methods for the use of chemically modifiable structures are incorporated into a biological polymer, such as DNA or RNA, such as dye clusters in DNA or RNA, to encode bits, wherein the clusters comprise sizes greater than those obscured by diffraction or other optical aberrations. Such embodiments, can allow for the data encoded in the polymer to be read without using single-molecule imaging methods, may enable using standard microscopy cameras or detectors to read the information encoded therein. In some embodiments, provided herein are methods and compositions comprising clusters which may enable writing and reading data at faster times with less expense. In some embodiments, such clusters are clusters of consecutively repeating units; the clusters comprise at least 10, 21, 625 units; the clusters comprise comprise at least about 0.6 or 1.2 kbp of repeating units; or the clusters define a data bit resolution defined by a distance of at least about 0.3, 0.6, or 1 microns about the length of the polymer, which permit for the data encoded in the polymer to be read standard microscopy cameras, without needing to control the forward or reverse movement of the polymer within a nanopore sequencer, and without computational analysis to account for diffraction. In some embodiments, with the use of clusters of chemically modifiable or modified structures, such difficulties may be obviated. In some embodiments, a stretch of polymer comprising iterative (e.g., iteratively spaced) modifiable residues can be written with variable resolution in order to overcome the issues presented by attempting to read and write at the single nucleobase level. In some embodiments, one focal point (e.g., region or spot) of light may comprise multiple modifiable dyes. In some embodiments, the stochastic variation of photomodulation may be averaged over multiple dyes, leading to fewer errors in writing.

In some embodiments, provided herein, compositions and methods may comprise iterative dyes along a polymer (e.g., DNA polymer strand). In such embodiments, methods comprising writing or reading data may comprise writing or reading of data wherein each bit comprises clusters of residues (e.g., residues labeled with dyes). In some embodiments, no exact positioning of the polymer relative to the light source may be required. In some embodiments, no exact positioning of the polymer relative to the light source may be required, as data can be encoded along the length of the polymer, wherein the encoding along the length of the polymer may start and end at any location in the polymer. In some embodiments, methods for reading or writing data may not comprise reversal of direction, wherein nanopore sequencing is used. In some embodiments, methods that do not comprise a step of reversing direction during nanopore sequencing may comprise reduced time for data writing. In some embodiments, no reversal of direction in nanopore sequencing may simplify the polymer design and the equipment needed.

In certain aspects, fluorescent-labeled noncovalent DNA assemblies may be built from synthetic oligonucleotides and assembled by hybridization. In such aspects, assemblies, built to be used as “molecular antennas”, may contain fluorescent labels along the assembled segments, and because each assembled unit is the same, no digital data may be encoded. Such a DNA assembly to encode digital information may comprise more than one DNA polymer and therefore digital data may not be stable due to degradation of the DNA assembly by de-hybridization. For example, when local modulation of fluorescence of a dye is utilized, information may be easily scrambled by de-hybridization/re-hybridization of the oligonucleotides contained in the assembly.

In order to permit for the use of fluorescent-labeled noncovalent DNA assemblies in the construction of data encoding polymers, provided herein are various compositions of polymers comprising photomodifiable groups. In some embodiments, provided herein are various methods comprising modification to photomodifiable groups comprising dyes. In some embodiments, polymers configured for encoding digital information may comprise clusters of photomodifiable groups.

In certain aspects, methods for encoding digital information on polymers may comprise synthesizing the polymer, unit by unit, with different monomers encoding binary bits. Such aspects may comprise the use of light to remove photocage groups. In certain aspects encoding occurs during the assembly of the polymer into a particular sequence encoding data.

In some embodiments, provided herein, methods may comprise the use of clusters of the same monomer to encode digital information. In some embodiments, provided herein, methods may comprise writing locally in the polymer already assembled. In some embodiments, provided herein, the polymer as constructed may not comprise encoding digital information, but rather may exist as an unwritten type of “tape”. In some embodiments, as provided herein, the polymer may be built far in advance of writing of data, and the polymer can be used to encode any arbitrary string of bits, by optical writing along the polymer strand. In some embodiments, such clusters are clusters of consecutively repeating units; the clusters comprise at least 10, 21, 625 units; the clusters comprise at least about 0.6 or 1.2 kbp of repeating units; or the clusters define a data bit resolution defined by a distance of at least about 0.3, 0.6, or 1 microns about the length of the polymer, which permit for the data encoded in the polymer to be read standard microscopy cameras, without needing to control the forward or reverse movement of the polymer within a nanopore sequencer, and without computational analysis to account for diffraction.

In certain aspects, compositions and methods may comprise incorporation of a photoswitchable fluorescent dye in synthetic DNA oligonucleotides. In certain aspects, the oligonucleotides may be constructed by chemical synthesis, and may be limited to a few dozen nucleotides in length. In certain aspects, the whole DNA (not locally) may be illuminated with light to change the color of the dye. In such aspects, no encoding of any digital information may occur. In such aspects, a fluorescent DNA base may not be incorporated into longer DNAs by polymerase enzymes.

In some embodiments, provided herein compositions and methods may comprise long polymers comprising iterative copies of a dye. In some embodiments, provided herein, compositions and methods may comprise the use of clusters of the dye to encode digital information. In some embodiments, provided herein, compositions and methods may comprise iterative long polymers comprising many copies of photomodifiable groups. In some embodiments, provided herein, compositions and methods may comprise optical encoding of digital data by converting clusters of such groups.

Turning now to the drawings and data, compositions and systems of data storage utilizing nucleic acid polymers, methods of incorporating chemically modifiable groups in polymers, and methods of encoding, in accordance with various embodiments, are disclosed. In several embodiments, a system of data storage comprises polymers that comprise a plurality of chemically modifiable structures along the nucleic acid polymer. Accordingly, a data encodable polymer is akin to a blank tape that is encodable, wherein the data encodable polymer is encoded by selectively modifying the modifiable structures in clusters along the polymer strand, which can be done by any writing method for modification, including (but not limited to) localized light or redox energy. In many embodiments, a cluster is a localized domain that is defined by the data encoding resolution; clusters will typically include two or more modified structures that have been modified in a writing step to encode a bit of data, each modified structure extending from the polymer. In some embodiments, the modified structures of a cluster have been uniformly altered such that all the chemically modifiable structures of the cluster undergo the same alteration. In some embodiments, only a portion of the modified structures of a cluster have been modified. In various embodiments, a chemically modifiable structure is a caging group, a quencher, a photobleachable fluorophore, or a photoconvertible fluorophore. Accordingly, in various embodiments, a chemically modifiable structure is modified by uncaging a nucleobase, uncaging a modified nucleobase, releasing a quencher, bleaching a fluorophore, or photoconverting a fluorophore. Modification of chemically modifiable structure in clusters can act as a data code, where a cluster of modified structures is akin to a data encoded “bit;” one modified structural state of the cluster is akin to a “0,” and a second modified structural state of the cluster is akin to a “1”. For instance, in one example, chemically modifiable structures can comprise a plurality of caged fluorophores and a plurality of quenched fluorophores provided in an equal ratio; releasing a cluster of only the plurality of quenchers of the cluster can be akin to “0” and releasing a cluster of the plurality of quenchers and uncaging the plurality caged fluorophores of the cluster can be akin to a “1”. It should be understood, however, that a binary code is not the only possibility, and data codes can be written in ternary, quaternary, or other numeral system code, which can be done utilizing multiple types of chemically modifiable structures or performing multiple writings/modifications. It should also be understood that spacing of unconverted and converted clusters can also encode data, as unconverted clusters can encode “zero” and converted clusters can encode “one”. The modification of a cluster of a plurality of chemically modifiable structures can be stable, or permanent, which allows for long-term archiving, especially if kept in a dark storage location.

In many embodiments, a polymer incorporates residues with a chemically modifiable structure incorporated into the backbone or attached thereupon, such that the polymer is synthesized with the chemically modifiable structures iteratively repeated along the polymer. In certain embodiments, the polymer is a nucleic acid polymer that incorporates nucleotides with a chemically modifiable structure attached thereupon, the chemically modifiable structures iteratively repeated along the polymer. In several embodiments, a polymer incorporates residues with a reactive group such that the residue with a reactive group is iteratively spaced along the polymer. The reactive group can be utilized to install chemically modifiable structures via a bonding reaction. In some embodiments, chemically modifiable structures are provided in pairs (e.g., each reactive group installs a pair, the pair consisting of two chemically modifiable structures). Likewise, in several embodiments, each chemically modifiable structure (or pair of chemically modifiable structures) can include a reactive group, which can be utilized to bond with one of the iteratively spaced reactive groups of the polymer such that each set can be installed. In many embodiments, the synthesis of polymer with the chemically modifiable structures or the installation of the chemically modifiable structures onto the polymer converts the polymer into a data encodable polymer capable of encoding data in clusters. In such embodiments, the clusters of modified structures are defined by the resolution of the data writing process. In some of such embodiments, 2 to 2000 contiguous chemically modifiable structures along the polymer are modified as a cluster, which is akin to a bit of data. In some embodiments, 2 to 20 modifiable structures are modified as a cluster. In some embodiments, 2 to 20 modifiable structures are modified as a cluster.

In some embodiments, when encoding data, contiguous chemically modifiable structures of the polymer are modified as a cluster such that the modifications within the cluster are uniform. In some embodiments, the contiguous chemically modifiable structures of the polymer are simultaneously modified, resulting in uniform modification within each cluster. In some embodiments, however, only a portion of the contiguous chemically modifiable structures of are modified when encoding data as clusters, resulting in clusters having at least one modified structure within the cluster.

Many embodiments are directed towards compositions of writable polymers. Any appropriate polymer can be utilized, including (but not limited to) biological polymers, organic polymers, and inorganic polymers. Biological polymers (and their analogues) include (but are not limited to) DNA, RNA, phosphorothioate DNA, glycerol nucleic acids (GNA), threose nucleic acids (TNA), 2′-fluoro-DNA 2′-O-methyl RNA, locked nucleic acids (LNA), peptide chains, and peptoid chains. A nucleic acid polymer may be single stranded or double stranded. Further, a nucleic acid polymer may utilize any enantiomer (e.g., d-DNA, 1-DNA).

In several embodiments, a data encodable nucleic acid polymer comprises a plurality of chemically modifiable structures that are linked by a polymer backbone. In some embodiments, when encoding data in clusters, portions of the polymer between the data encoded clusters are left unmodified to provide a space between the clusters. In some embodiments, portions of the data polymer are unmodifiable, which can be utilized space between clusters that remains unmodified. In various embodiments, a data encodable nucleic acid polymer can further include delimiters and/or data tags for labeling or locating the data.

In several embodiments, a data encoding procedure is utilized to encode a data encodable polymer with data. Data encoding can be performed by selectively and locally modifying the chemically modifiable structures of a polymer in clusters such that the encoded polymer contains a sequence of clusters with structural modifications, akin to a binary code of “zeros” and “ones”.

In accordance with many embodiments, data encoded polymers are stored in the dark free of photobleaching light. In some embodiments, data encoded polymers are stored in environments that exclude air or oxygen, which may enhance stability. Stabilizers such as (for example) alcohol, antioxidants, chelating agents and biological inhibitors (e.g., nuclease inhibitors), may be included with the stored polymer. To read the data on encoded nucleic acid polymers, a nanopore capable of analyzing structural differences in monomers can be utilized, such as Oxford Nanopore Technologies PromethION, MinION, and GridION sequencing platforms (Oxford, UK). Also, a nanopore device can be fabricated or manufactured for reading the data. The nanopore can be comprised of solid-state materials, or can contain one or more proteins. Alternatively, when fluorophores are utilized within chemically modifiable structures, any appropriate nanopore capable of detecting fluorescence of the fluorophores can be utilized, such as Pacific Bioscience's Single Molecule, Real-Time (SMRT) sequencing platform (Menlo Park, CA), or nanopores with plasmonic features for focusing light energy near the pore.

In many embodiments, the chemically modifiable structures in the data encodable polymer are convertible into two different states that can define one or zero in the digital sense. An encoded cluster is distinct from the unwritten state, and the difference is detectable, in accordance with various embodiments, via optical differences, differences in current, or differences in redox state, or differences in magnetic state. In some embodiments, a string of encoded clusters along the encoded polymer strand provides a string of digital data. In some embodiments, the resolution of the method to encode data defines the cluster size. The various methods of this disclosure can be carried out with polymers that are considerably longer than can be assembled in the bottom-up approach, thus providing greater density of data storage. In addition, the data encoding can be carried out more rapidly than the bottom-up approach, since the bits can be written at high speeds and do not require chemical assembly of molecules during the encoding process.

Generation of Polymers with Chemically Modifiable Structures

Compounds and methods of synthesis in accordance with embodiments of the disclosure are based on generating polymers having iterative residues with chemically modifiable structures to generate a data encodable polymer. A contiguous portion of a plurality of chemically modifiable structures along the polymer is encoded as a cluster and utilized as a data “bit,” such that polymer can be encoded in a code (e.g., binary code). Each cluster of modified structures (i.e., bit) can exist in two or more states, a first written state, and at least a second written state. In some embodiments, a first written state is akin to a “zero” in binary code and a second written state is akin to “one.” Data encodable polymers can be generated having long lengths (e.g., 5 to 50,000 residues with chemically modifiable structures, or more) and can be produced in bulk, prior to data encoding.

In several embodiments, a data encodable nucleic acid polymer comprises a sequence of nucleotides that are linked by the polymer backbone. In many embodiments, the sequence comprises iteratively repeated nucleotides with chemically modifiable structures. For example, a sequence can be generated in which each thymine (T) has a chemically modifiable structure; the thymines can be iteratively repeated with regularity. It should be understood that the plurality of chemically modifiable structures can be extended from any nucleotide structure as long as the nucleotide is provided in an iterative repeated fashion. In various embodiments, nucleotide structures adducted with a chemically modifiable structure are repeated every other nucleotide, every 3^{rd} nucleotide, every 4^{th} nucleotide, every 5^{th} nucleotide, or in any other iterative repeat. It is to be understood, however, that perfect regularity is not essential, and nucleotide structures adducted with a chemically modifiable structure are irregularly repeated.

In several embodiments, the chemically modifiable structures are designed to be alterable by light pulses or by redox signals. In certain embodiments, the chemically modifiable structures are encoded as a cluster in accordance with a spatial resolution. The spatial resolution depends, at least in part, on the data encoding mechanism. For instance, if an optical light source and device with 20 nm of resolution is used to modify the chemically modifiable structures, then the data encoded cluster can be approximately 59 bp, which is approximately 20 nm in length. This is smaller than the wavelength of the light, but subwavelength resolution can be achieved by use of methods such as plasmonics, zero mode waveguides, or laser techniques such as STED. Furthermore, when the light source is impinged on the polymer, all the chemically modifiable structures within the spatial resolution can be modified simultaneously, and thus encoding a data bit cluster. The lower the resolution of the localized energy, the more modified structures are included in an encoded cluster. Conversely, the higher the resolution, the fewer modified structures are included in an encoded cluster. In some embodiments, encoded clusters are separated by stretches of the polymer that remain unmodified, and thus can contain chemically modifiable structures in their original, unmodified state. In some embodiments, residues lacking chemically modifiable structures can be utilized as spacers between clusters. In various embodiments, a data encodable polymer can further include delimiters and/or data tags for labeling the data, each of which can be provided by a particular sequence of residues.

In many embodiments, data is encoded in clusters of modified structures along a nucleic acid polymer that have been modified by light or redox. The starting state and the encoded state can be distinguished by their altered properties in the polymer. For example, chemically modifiable structures may have a change in fluorescence intensity or wavelength, a change in structure that altered size/shape so that they can be distinguished by nanopore current, a change in magnetic or spin properties, or a change in redox properties. For encoding data in binary form, in accordance with some embodiments, the chemically modifiable structures can be changed from a modifiable structure to a first modified structure (akin to 0) and/or to a second modified structure (akin to a 1). This may be achieved by including two chemically modifiable structures attached to unique residue structures along a nucleic acid polymer (e.g., one structure extending from a T and second structure extending from a C), or two chemically modifiable structures that are linked and attached via one attachment point (e.g., both structures extending from a T). An example of this case might include photobleaching of two differently-colored fluorescent dyes attached to the same nucleotide. In some embodiments, a chemically modifiable structure is a single molecule that can be converted from a starting state into two different structural outcomes, such as through photoconversion of cyanine dyes. In some embodiments, another molecular approach is to convert two different chemically modifiable structures into new chemical groups that can be each independently detected, as by fluorescent labels or redox groups. In several embodiments, the assembled data encodable polymer contains such convertible structures all along the strand, to ensure that a bit can always be encoded regardless of the polymer's position relative to the writing device.

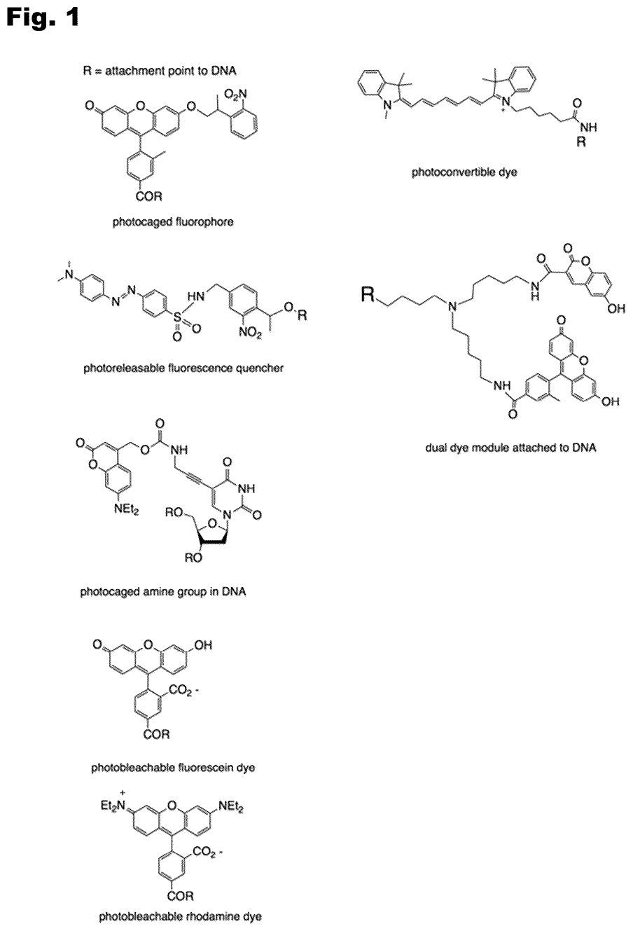

FIG. 1 provides examples of chemically modifiable structures that can be attached to a residue and incorporated into a polymer. The chemically modifiable structures enable the polymer to be data encodable by localized light or redox signals. A chemically modifiable structure, in accordance with various embodiments, comprises one or more chemical groups that are capable of being structurally altered from a first structure state (e.g., caged) into a second structural state (e.g., uncaged) by controlled reaction chemistry (e.g., light energy or redox energy). In some embodiments, a chemically modifiable structure incorporates a fluorophore that is caged, where the caged fluorophore is a first structural state and release of the cage results in a second structural state. In some embodiments, a chemically modifiable structure incorporates a quenched fluorophore, where the quenched fluorophore has a first structural state and release of the quencher results in a second structural state, usually resulting in brighter fluorescence. In some embodiments, a chemically modifiable structure incorporates a fluorophore capable of being converted from a first state having a particular fluorescent quality (e.g., a first emission or absorption wavelength) into a second state having a second distinguishable fluorescent quality (e.g., a second emission or absorption wavelength). In some embodiments, a chemically modifiable structure incorporates two structurally alterable groups to provide a set of structures that is modifiable into at least two different states.

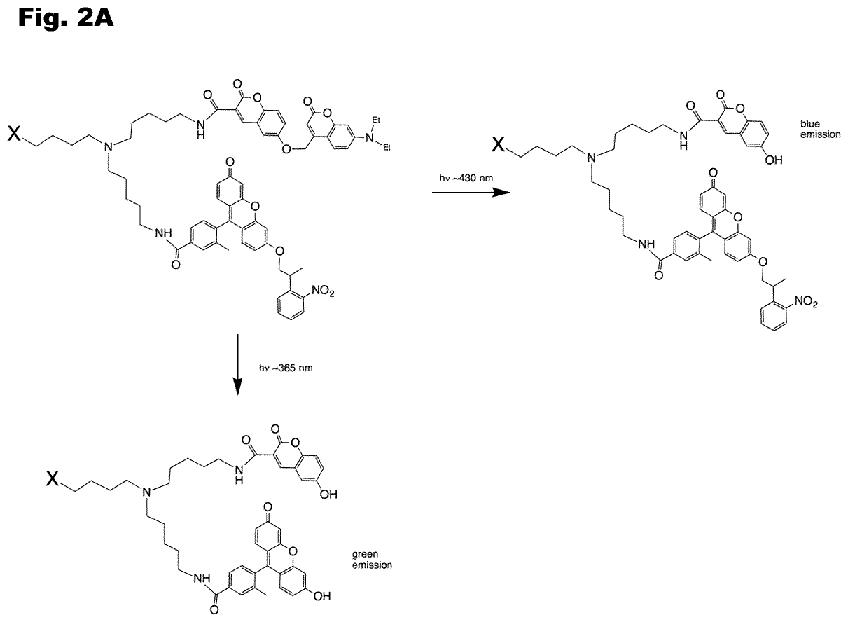

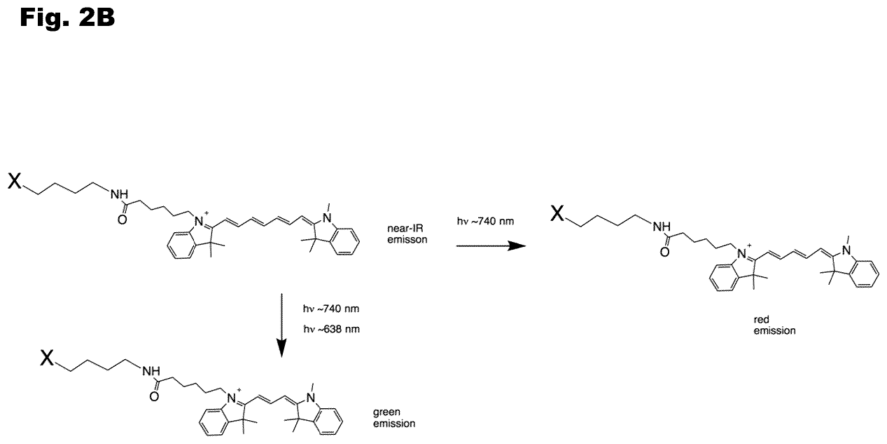

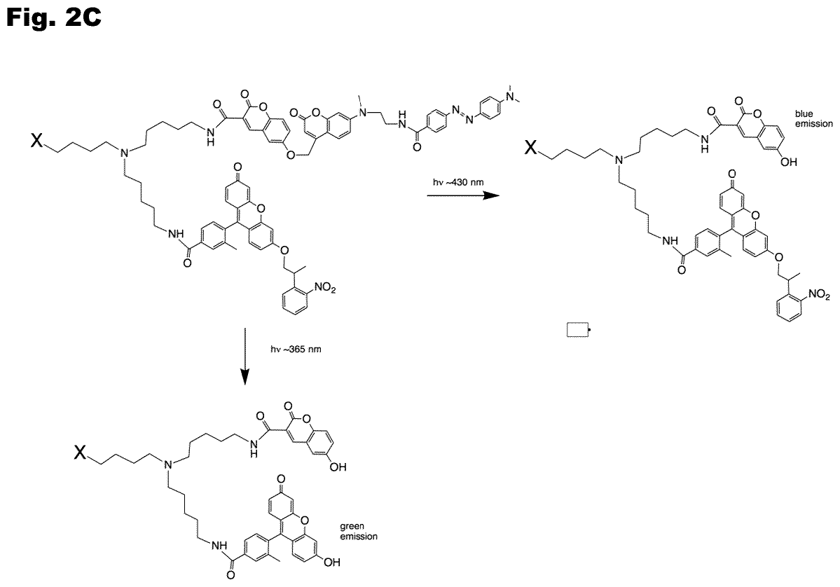

FIGS. 2A to 2C each provide an example of sets of chemically modifiable structures that can be utilized to encode data, in accordance with various embodiments. In many embodiments, the chemically modifiable structures can be incorporated along the polymer strand. As shown in FIGS. 2A to 2C, these chemically modifiable structures have the capacity for dual bit writing. Accordingly, each set of chemically modifiable structures is added to a residue in an unwritten state and then converted into a first written state and/or a second written state. The “X” denotes the attachment with the polymer residue.

The example in FIG. 2A is a set of chemically modifiable structures that comprises two caged fluorophores. In some embodiments, the two-fluorophore adduct comprises two fluorophores within approximately 30 angstroms of one another. In this example, two fluorophores of distinct color are utilized in the set of chemically modifiable structures: coumarin and Tokyo green. In addition, each fluorophore is caged with a caging group. Before data writing, the polymer has little or no fluorescence emission due to the caging of the fluorophores. To encode a bit of data, one or more pulses of violet light (e.g., approximately 430 nm) light is employed to singularly uncage a blue coumarin fluorophore, resulting in blue fluorescence. Alternatively, one or more pulses of UV light (e.g., approximately 365 nm) is utilized to uncage both the blue coumarin fluorophore and the Tokyo green fluorophore, resulting in a coumarin blue plus green Tokyo green emission signal. Or alternatively, dual pulses of violet light (e.g., approximately 430 nm) and UV light (e.g., approximately 365 nm) uncage both the blue coumarin fluorophore and the Tokyo green fluorophore, resulting in a coumarin blue plus green Tokyo green emission signal. When both fluorophores are uncaged, the blue fluorophore can donate energy to the green fluorophore, resulting in Forster energy transfer, enhancing the green florescence and decreasing the blue florescence. Thus, in this approach, data can be encoded as a binary code of clusters of blue and green emission signals in sequence along the polymer. The set of chemically modifiable structures can be installed onto a nucleotide or synthesized as part of the nucleotide and then incorporated with the polymer via biological or chemical synthesis. Alternatively, the set of chemically modifiable structures can be installed onto the polymer via a reactive residue attached to the backbone, as depicted in the figures by the X. While a few examples are provided, it is understood that any appropriate fluorophores and cages may be used in accordance with the various embodiments.

Various photo removable groups can be incorporated into caged fluorophores (see, e.g., Y. Zhao, et al., J Am Chem Soc. 2004; 126:4653-63; the disclosure of which is incorporated herein by reference). As can be seen in the figures, each fluorophore has a caging constituent that is linked by a linker (e.g., ether or carbonate or carbamate group) that is cleavable with energy (e.g., light or redox). The fluorophores can be attached directly to the polymer backbone via a reaction group on a residue attached to the backbone. While a few examples are provided, it is understood that any appropriate photo-removable group and fluorophore may be used in accordance with the various embodiments.

The example in FIG. 2B is a set of chemically modifiable structures that comprises a photoconvertible fluorophore in which light at particular wavelengths can convert the fluorophore to emit a second and/or third fluorescent wavelength. In several embodiments, a cluster of photoconvertible fluorophores can be utilized in manner akin to “bits” of data, enabling conversion from a first structural state (e.g., first emission wavelength) to a second structural state (e.g., second emission wavelength), akin to “0” or “1” digital bit designations. In some embodiments, a photoconvertible fluorophore can be converted from a first emission state (e.g., first emission wavelength) to a second emission state (e.g., second emission wavelength) and further to a third emission state (e.g., third emission wavelength), which allows a ternary code or positively written binary code (i.e., the first emission state is unwritten, the second emission state is akin to “0” and the third emission state is akin to “1”). The fluorescent state of the bit is detectable utilizing a nanopore device capable of detecting fluorescence of individual fluorophore molecules.

Various photoconvertible fluorophores groups can be utilized (see, e.g., T. J. Chozinski, L. A. Gagnon, and J. C. Vaughan, FEBS Lett. 2014; 588:3603-12; the disclosure of which is incorporated herein by reference). As can be seen in the example of FIG. 2B, a heptamethine cyanine dye can be converted to either a pentamethine cyanine dye or a trimethine cyanine dye by pulses of specific wavelengths of light. In this example, the heptamethine cyanine dye having a 7-carbon alkene-containing chain emits near-IR fluorescence. Light energy at approximately 740 nm modifies the alkene-containing chain of the heptamethine cyanine dye resulting in pentamethine cyanine dye having a 5-carbon chain. The pentamethine cyanine dye emits red fluorescence. Furthermore, light energy at approximately 638 nm modifies the alkene-containing chain of the heptamethine cyanine dye or pentamethine cyanine dye resulting in trimethine cyanine dye having a 3-carbon chain. The trimethine cyanine dye emits yellow/green light. Thus, in this approach, data can be encoded as a binary code of red and yellow/green emission signals. The set of chemically modifiable structures can be installed onto a nucleotide or synthesized as part of the nucleotide and then incorporated with the polymer via biological or chemical synthesis. Alternatively, the set of chemically modifiable structures can be installed onto the polymer via a reactive residue attached to the backbone, as depicted in the figures by the X. While a few examples are provided, it is understood that any appropriate photoconvertible fluorophore may be used in accordance with the various embodiments.

The example in FIG. 2C is a set of chemically modifiable structures that comprises a combination of a releasable quencher and a caged fluorophore. In some embodiments, the two-fluorophore bit comprises two fluorophores within approximately 30 angstroms of one another. In this example, two fluorophores of distinct color are utilized in the set of chemically modifiable structures: unmodified coumarin and the photocaged Tokyo green. In addition, a photoreleasable fluorescence quencher with a photocleavable linker can be incorporated near the two dyes (and in this example is attached to the coumarin blue fluorophore). Before data writing, the polymer has little or no fluorescence emission due to the fluorescence quencher and the caging of the fluorophore. To write a bit of data, one or more pulses of violet light (e.g., approximately 430 nm) light is employed to singularly release the quencher, resulting in blue fluorescence from the blue coumarin. Alternatively, one or more pulses of UV light (e.g., approximately 365 nm) is utilized to release the quencher and uncage the Tokyo green fluorophore, resulting in a coumarin blue plus green Tokyo green emission signal. Or alternatively, dual pulses of violet light (e.g., approximately 430 nm) and UV light (e.g., approximately 365 nm) release the quencher and uncage the Tokyo green fluorophore, resulting in a coumarin blue plus green Tokyo green emission signal. When both fluorophores are unquenched and uncaged, the blue fluorophore can donate energy to the green fluorophore, resulting in Forster energy transfer, enhancing the green florescence and decreasing the blue florescence. Thus, in this approach, data can be encoded as a binary code of blue and green emission signals in sequence along the polymer. The set of chemically modifiable structures can be installed onto a nucleotide or synthesized as part of the nucleotide and then incorporated with the polymer via biological or chemical synthesis. Alternatively, the set of chemically modifiable structures can be installed onto the polymer via a reactive residue attached to the backbone, as depicted in the figures by the X. While a few examples are provided, it is understood that any appropriate fluorophores and cages may be used in accordance with the various embodiments.

Various quencher groups can be utilized (see, e.g., J. R. Lakowicz, (Ed.). (2013). Principles of fluorescence spectroscopy. Springer science & business media. pp. 277-330 “Quenching of Fluorescence”; and M. K. Johansson, Methods Mol Biol. 2006; 335:17-29; the disclosures of which are each incorporated herein by reference). As can be seen in the figures, a quencher has a linker (e.g., nitrobenzyl group) that is cleavable with light energy. The quencher can be attached directly to the polymer backbone, to a residue attached to the backbone, or to a fluorophore. While a few examples are provided, it is understood that any appropriate quencher, releasing mechanism, and fluorophore may be used in accordance with the various embodiments.

Several embodiments are directed to encodable polymers. In many embodiments, an encodable polymer comprises a plurality of chemically modifiable structures that are iteratively repeated. In several embodiments, the plurality of chemically modifiable structures along the polymer strand are modified in clusters, such that each cluster is one bit of encodable data. In many embodiments, a cluster is defined by the resolution of the data encoding device. For instance, if the data encoding device has a resolution of 20 nm, then each cluster is defined to be 20 nm in length, which is approximately 59 bp. In various embodiments, a cluster is defined to be any length between 1 nm and 400 nm. In certain embodiments, a cluster is defined to be 5 nm, 10 nm, 15 nm, 20 nm, 25 nm, 30 nm, 35 nm, 40 nm, 45 nm, 50 nm, 75 nm, 100 nm, 125 nm, 150 nm, 175 nm, 200 nm, 225, nm, 250 nm, 275 nm, 300 nm, 325 nm, 350 nm, 375 nm, or 400 nm in length. Further, a cluster can incorporate any plurality of modified structures, dependent on the iterative spacing of the structures and the data encoding spatial resolution. In certain embodiments, the iterative spacing of the chemically modifiable structures is every other residue, every 3^{rd} residue, every 4^{th} residue, every 5^{th} residue, every 6^{th} residue, every 7^{th} residue, every 8^{th} residue, every 9^{th} residue, every 10^{th} residue, every 11^{th} residue, every 12^{th} residue, every 13^{th} residue, every 14^{th} residue, every 15^{th} residue, every 16^{th} residue, every 17^{th} residue, every 18^{th} residue, every 19^{th} residue, every 20^{th} residue, every 21^{st} residue, every 22^{nd} residue, every 23^{rd} residue, every 24^{th} residue, every 25^{th} residue, or any other appropriate spacing as appropriate to the data encoding spatial resolution. In certain embodiments, the chemically modifiable structures are irregularly repeated.

In various embodiments, polymers incorporating chemically modifiable structures can be any length, for example, from as short as 15 residues to longer than 100,000 residues. In certain embodiments, a polymer is greater than 100 residues long, is greater than 200 residues, is greater than 300 residues, is greater than 400 residues, is greater than 500 residues, is greater than 1000 residues, is greater than 5000 residues, is greater than 10,000 residues, is greater than 50,000 residues, or is greater than 100,000 residues. Maximum lengths are only limited by the stability of the polymer, by the method used to synthesize the polymer, and by the method used to read the encoded data. Longer strands containing more bits have the advantage of containing more data per molecule.

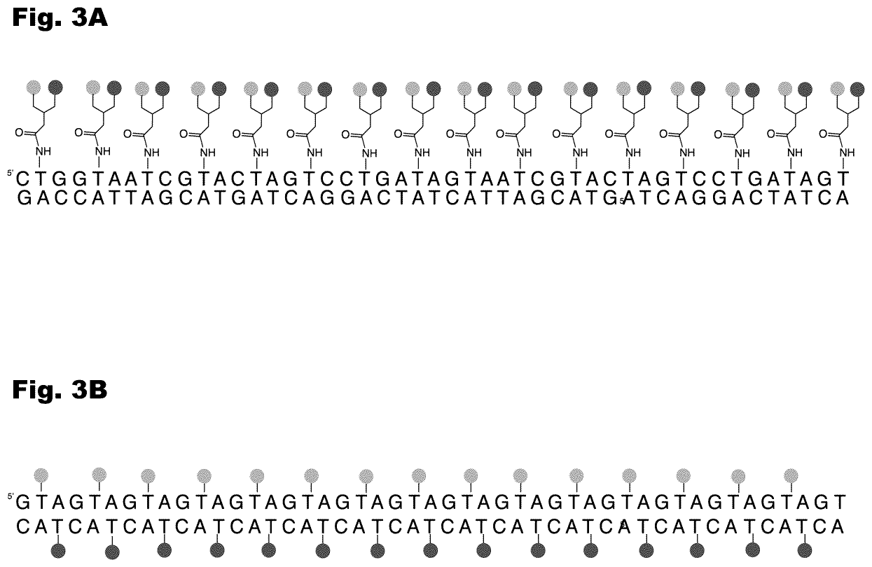

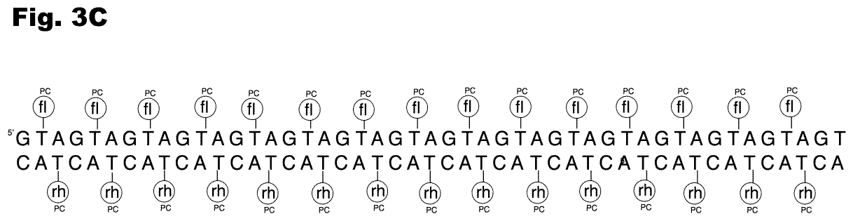

Provided in FIG. 3A is an exemplary schematic of a nucleic acid comprising chemically modifiable structures along the polymer. In this example, the polymer comprises adducts that include two chemically modifiable structures (e.g., green and red dyes as shown) that are each attached to thymine and repeated every 3^{rd} nucleotide on one strand of a double stranded polymer. Although adducts are shown to be extended from thymine, the adducts can be extended from any nucleotide, including (but not limited to) thymine (T), guanine (G), cytosine (C), adenine (A), uracil (U), or any other residue capable of being polymerized into a nucleic acid polymer. Further, although adducts are shown to be extended every 3^{rd} nucleotide, adducts can be iteratively spaced in any fashion such that at least a plurality of contiguous adducts are modified in clusters by the data encoding mechanism.

Provided in FIG. 3B is an exemplary schematic of a nucleic acid comprising chemically modifiable structures along the polymer. In this example, the polymer comprises adducts that include a single chemically modifiable structure (e.g., green dye or red dye) that are each attached to thymine and repeated every 3^{rd} nucleotide; the adducts of the top strand incorporate a green dye chemically modifiable structure and the adducts of the bottom strand incorporate a red dye chemically modifiable structure.

Provided in FIG. 3C is an exemplary schematic of a nucleic acid comprising chemically modifiable structures along the polymer. In this example, the polymer comprises adducts that include a single chemically modifiable structure that are each attached to thymine and repeated every 3^{rd} nucleotide; the adducts of the top strand incorporate a photocaged fluorescein dye chemically modifiable structure and the adducts of the bottom strand incorporate photocaged rhodamine dye as the chemically modifiable structure.

Numerous embodiments are also directed to a nucleic acid polymer further incorporating one or more of spacers, delimiters, and data tags. In accordance with various embodiments, a spacer is a residue incorporated within a polymer that provides a space between chemically modifiable structure bits. In some embodiments, a data encodable nucleic acid polymer will utilize the same residue structure repeatedly for each and every spacer. In some embodiments, however, a nucleic acid polymer will utilize two or more different residues as spacers. Any appropriate residue may be utilized as spacers, including naturally occurring nucleobases, unnatural nucleobases, tetrahydrofuran abasic residues, and/or ethylene glycol residues.

A delimiter, in accordance with various embodiments, is a residue that signifies a boundary. In some embodiments, a delimiter is utilized to separate two adjacent data fields. Any appropriate residue lacking ability to install bits may be utilized as a delimiter, including naturally occurring nucleobases, unnatural nucleobases, tetrahydrofuran abasic residues, and/or ethylene glycol residues.

In several embodiments, a data tag is a string of monomers (typically 4 or more residues) that signifies certain data. For instance, a data tag can signify type of data, date, data source, or any other information. Any appropriate residues lacking ability to install bits may be utilized as data tag residues, including naturally occurring nucleobases, unnatural nucleobases, tetrahydrofuran abasic residues, and/or ethylene glycol residues.

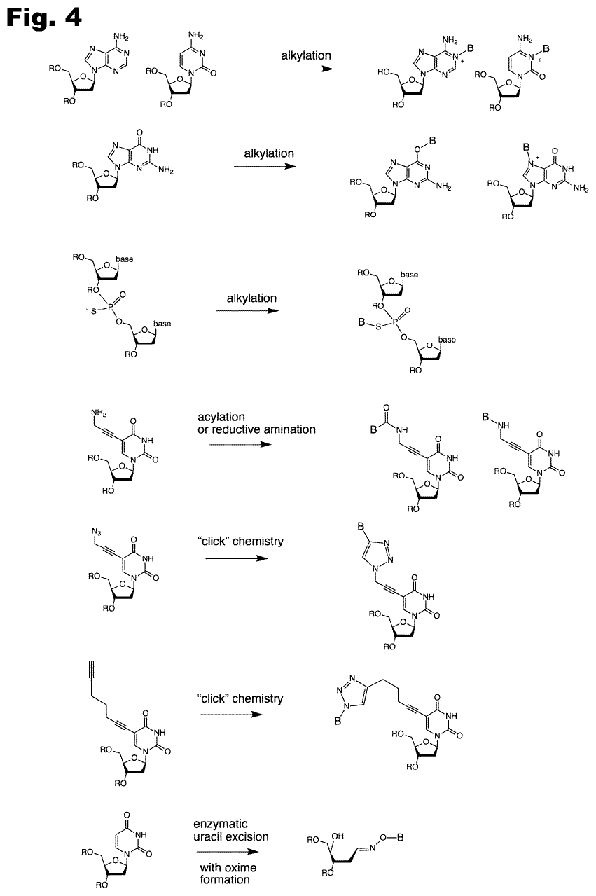

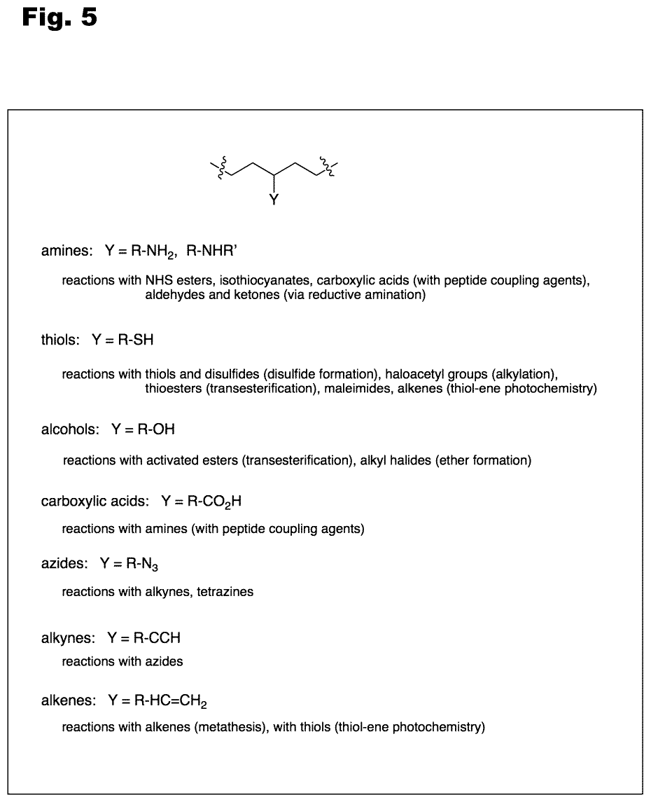

In some embodiments, an existing nucleic acid molecule can be utilized as a substrate to add chemically modifiable structures. In some embodiments, nucleotides having chemically modifiable structures are incorporated into a polymer. In some embodiments, chemically modifiable structures are installed onto a nucleobase and/or an existing nucleic acid molecule onto by a chemical reaction with nucleobase (e.g., see FIG. 4). In some embodiments, polymerase extension is performed incorporate residues with a reactive functional group onto a nucleic acid polymer (e.g., see FIG. 5). In some embodiments, polymerase extension is performed to incorporate residues with chemically modifiable structures already attached or synthesized within the residues. Any appropriate means to perform polymer extension can be utilized. In several embodiments, when performing polymer extension, one or more nucleoside triphosphates are modified with a functional group or chemically modifiable structure such that the functional group or chemically modifiable structure is incorporated into the nucleic acid polymer throughout the polymer chain. Incorporation of reactive functional groups permits installation of sets of chemically modifiable structures at each of the functional groups along the polymer.

### Modes of Data Encoding and Decoding

Several embodiments are directed towards encoding and decoding data on polymers utilizing chemically modifiable structures. In many embodiments, a data encodable polymer is provided having chemically modifiable structures iteratively repeated along the polymer. The provided writable polymer may also have spacers, delimiters, and data tags, as described herein. To write data upon a polymer, in accordance with various embodiments, an individual strand is passed through a device having a nanopore. Various nanopore devices can enable the structural changes of the chemically modifiable structures as they pass through the pore (see, e.g., D. Garoli, et al., Nano Lett. 2019; 19:7553-7562; and M. Rahman, et al., Lab Chip. 2021; 21:3030-3052; the disclosures of which are each incorporated herein by reference). Photoexcitation of photocages or cleavable linkers progressively along the strand results in breaking chemical bonds to yield structural modifications. Such devices can include structures near the nanopore that amplify light energy or redox energy to highly localized positions. Examples include (but are not limited to) plasmonic amplifiers such as metallic bowties, metallic nanorods, or zero mode waveguides (see, e.g., J. D. Spitzberg, et al., Adv Mater. 2019; 31:e1900422, the disclosure of which is incorporated herein by reference).

Pulsing energy on such plasmonic structures can yield variable resolutions, from as small as one nanometer up to as large as the wavelength of the light. Alternatively, the use of two lasers can be employed via the STED technique to achieve highly localized illumination (see, e.g., S. J. Sahl and S. W. Hell High-Resolution 3D Light Microscopy with STED and RESOLFT. 2019 Aug. 14. In: Bille J F, editor. High Resolution Imaging in Microscopy and Ophthalmology: New Frontiers in Biomedical Optics. Chain (CH): Springer; 2019. Chapter 1, the disclosure of which is incorporated herein by reference). Any resolution can be utilized, as achievable by the device and as desired for cluster size. In several embodiments, the cluster size is defined by the data encoding device resolution. In many embodiments, the resolution of the data encoding device is determined by a preferred cluster size.

In several embodiments, the act of encoding is achieved by passing the polymer through the plasmonic nanopore and using pulses of light of two wavelengths to convert two different groups into one or zero states. In many embodiments, the encodable polymer is stretched and a subwavelength focusing technique, such as STED, is used to locally impinge light of two different wavelengths on the chemically modifiable structures. In various embodiments, a contiguous string of chemically modifiable structures will fall within the resolution of the light energy, and will then be uniformly converted as a group since they all receive the same light pulse, resulting in the encoding of a cluster bit. Other methods of moving DNA past a writing mechanism, such as flowing stretched DNAs in a capillary, are also contemplated.

It is understood that the modified structures within a cluster that have undergone modification may be completely and uniformly modified. For example, if ten green dyes and ten red dyes all occur within the resolution of a single energy pulse having 20 nm resolution, and an encoding of a “one” is done by bleaching the green dyes, a pulse of light might convert all ten green dyes in that spatial resolution to a dark state, revealing a purely red fluorescence signal at that spot. In some embodiments, however, a pulse that converts only 50% of those green dyes to a dark state still provides encoded data, since this yields a detectable difference in fluorescence (a red to green ratio of 2:1). Thus, in accordance with various embodiments, any detectable change within an encoded cluster can be utilized as encoded data. In some embodiments, data encoding results in at least one modification of the chemically modifiable structures within the encoding resolution. In some embodiments, data encoding results in at least a plurality of modifications of the chemically modifiable structures within the encoding resolution.

In some embodiments, modification of chemically modifiable structures is not required to be performed at every repeating unit of the polymer. Units may be skipped during the encoding process, resulting in spaces between encoded clusters. When decoding polymers with skipped units, such skipped units can be interpreted as blank or null and the reader progresses to the next fluorescent position to find the next cluster bit in the string. Skipping of a repeating unit during encoding and decoding may be performed intentionally to space out bit encoding to best suit the resolution of the encoding method; skipping may also occur in random fashion due to stochastic movements and alteration of the rate of the polymeric molecule passing through the pore.

In many embodiments, the encoding device is provided a software code for encoding the data into the polymer. Accordingly, the encoding device, directed by this code, will control pulses of energy by time and/or wavelength to selectively modify the chemically modifiable structures of the polymer to yield a data code (e.g., binary code). After encoding a code of data into the polymer, it can be stored in the dark and by any appropriate means for maintaining integrity of the polymeric molecule. For instance, data encoded polymers can be stored dry, as a precipitate, or in an appropriate solution at room temperature, or at colder temperatures (e.g., −20° C.). Stabilizers such as (for example) alcohol, antioxidants, chelating agents and biological inhibitors (e.g., nuclease inhibitors or protease inhibitors), may be included with the stored polymer.

Polymers most efficiently store data at the single molecule level, providing the highest potential density of information. In some embodiments, however, if redundancy of data is required for better accuracy of data storage, then a plurality of polymers could be used to redundantly encode the same data on each polymer of the plurality. Error correction algorithms are already well developed for digital data storage, and some of these algorithms can be applied in the present approach (see J. Li, et al., IEEE Transactions on Emerging Topics in Computing. 2021; 9:651-663, the disclosure of which is incorporated herein by reference).

Highly localized light excitation can be achieved via specialized sub-wavelength microscopic focusing strategies such as STEDX, or by the use of nanoplasmonic structures such as bow ties or by the use of zero-mode waveguides (see Y. Fang and M Sun, Light Sci Appl. 2015; 4:e294; and X. Shi, et al. Small. 2018; 14:e1703307; the disclosures of which are each incorporated herein by reference). Timing of energy pulses and controlled passage of the writable polymer can be in concert with appropriate spacing such that data is encoded with fidelity.

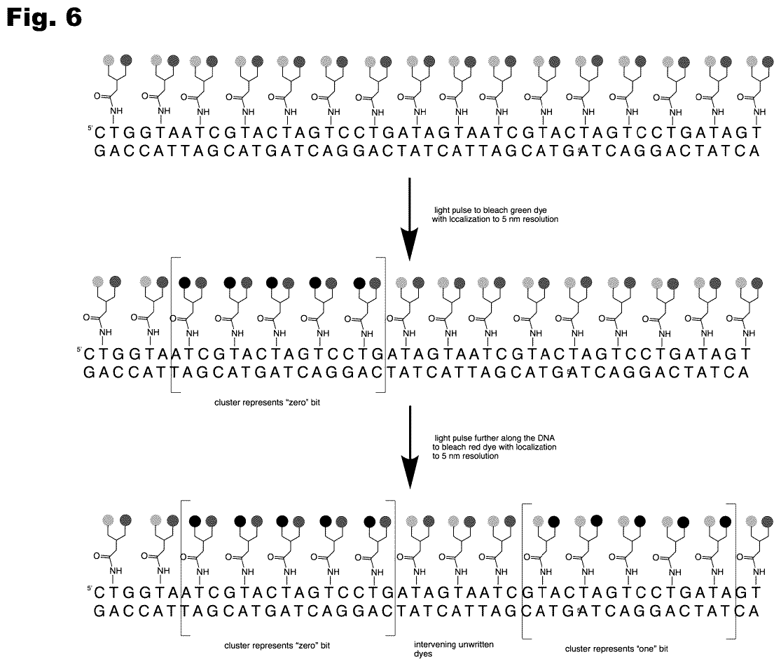

Provided in FIG. 6 is a schematic of a method to encode a data encodable nucleic acid molecule via an encoding device with a resolution that yields clusters of modified structures. In this example, the nucleic acid polymer is constructed to contain a sidechain at thymidine residues, where the sidechain carries fluorescein (green) and rhodamine (red) dyes linked to an amine group on thymine bases. To encode data, the nucleic acid molecule can be passed a plasmonic nanopore having optical resolution of 5 nm. As the DNA is passed through the pore, the fluorescein (green) and rhodamine (red) dyes within a cluster can be modified into a written state, encoding a “zero” or “one.” As shown in FIG. 6, a pulse of light at a wavelength to excite fluorescein is impinged on the plasmonic structure, resulting in focusing high energy on a cluster of five chemically modifiable structures. The light energy bleaches the fluorescein in the cluster, yielding a encoded cluster of rhodamine fluorescence, with fluorescein fluorescence diminished. Further, as the nucleic acid passes through the nanopore, a pulse of light at a wavelength to excite rhodamine is impinged on the plasmonic structure, resulting in focusing high energy on a cluster of five chemically modifiable structures. The light energy bleaches the rhodamine in the cluster, yielding an encoded cluster of fluorescein fluorescence and diminished rhodamine fluorescence. By strategically photobleaching fluorescein or rhodamine dyes, a binary code can be written, as denoted by the green (fluorescein) and red (rhodamine) clusters. Unwritten portions (here shown in FIG. 6 as green and red fluorescence) can be left between the encoded clusters, providing a spacer between the data encoded clusters.

In several embodiments, an encoded polymer comprises clusters of converted structures in the “one” or “zero” state, with intervening unencoded (unconverted) structures between the encoded clusters. In many embodiments, reading the clusters is done by observing the string of “one” and “zero” state clusters along the polymer. The observation can be done by passing the polymer through a plasmonic nanopore and observing the changed fluorescence in the encoded bits as the polymer strand passes. Alternatively, the polymer can be passed through a zero-mode waveguide in a nanowell and imaged in time, as used in SMRT sequencing. Another approach is to stretch or comb the polymer strand and image fluorescence changes along the linear stretched strand.

In many embodiments, to decode the data on encoded nucleic acid polymers, any appropriate nanopore capable of reading fluorescence of single fluorophores or capable of analyzing structural differences can be utilized. In certain embodiments, a device is capable both of encoding and decoding nucleic acid polymers. In certain embodiments, a single nanopore has dual functionality for both encoding and decoding polymers, however, some devices may include distinct nanopores for performing encoding and decoding. Various nanopore devices are available commercially, or alternatively, a nanopore device can be fabricated or manufactured for encoding and/or decoding the data. The nanopore can be comprised of solid-state materials.

An alternative to utilizing a nanopore device for decoding, a polymer may be stretched and imaged utilizing a device capable of decoding fluorophores along stretched polymers. Polymer (especially nucleic acid) stretching or combing methods are known to practitioners of the art (see, e.g., Z. E. Nazari and L. Gurevich, J. Self-Assembly and Molecular Electronics 2013; 1:125-148; A. Kaykov, et al., Sci Rep. 2016; 6:19636; the disclosures of which are incorporated herein by reference). Alternatively, in some embodiments, superresolution microscopy can be employed (via the STED approach or other known methods) for achieving high spatial resolution. Imaging of a single polymer strand by STED can yield a sequence of dye colors, which represents a string of bits. The method can be automated for high throughput, with many strands stretched in one field of view, and automated imaging software that reads a string of dyes (bits) and converts it into digital information.

The use of energy pulses to convert or otherwise alter dyes and/or quenchers in a polymer results in detectable structural changes in the repeating units of the polymers. Accordingly, in addition to the above fluorescence methods, data encoded polymers can also be read by non-fluorescence methods. An example includes nanopore sequencing by the use of alterations of ion flow. Another method involves the reading of optical absorption or other spectral signatures in the altered dyes (such as vibrational modes) as the strand passes though the pore. Another method involves the reading of redox signals as the strand passes through the pore.

In certain aspects, provided herein are various methods of encoding data onto a writable polymer, comprising: providing a data encodable nucleic acid polymer having a sequence of nucleotides in which one or more chemically modifiable structures are iteratively repeated, wherein each chemically modifiable structure of the one or more chemically modifiable structures are attached onto the nucleic acid polymer; and wherein the one or more chemically modifiable structures are capable of being modified into a second structural state by pulses of light energy or of redox energy; and selectively modifying, utilizing a data encoding device, a subset of the one or more chemically modifiable structures along the nucleic acid polymer into the second structural state such that a data encoded polymer is generated as defined by the modified structures within a plurality of clusters, and wherein the length of each cluster of the plurality of clusters is determined by a spatial resolution.

In some embodiments, the spatial resolution is defined by the resolution of a laser (e.g., laser spot size or laser spot). In some embodiments, the spatial resolution comprises the minimum length of the polymer that comprises one bit (e.g., a single bit). In some embodiments, the spatial resolution comprises the length of the polymer that can be written as a single bit. In some embodiments, the spatial resolution minimum comprises the length of the polymer that can be written as a single bit. In some embodiments, the spatial resolution comprises the length of the polymer that can be written as a single bit.

In some embodiments, each chemically modifiable structure of the one or more chemically modifiable structures comprises a photobleachable fluorophore.

In some embodiments, the selectively modifying comprises photobleaching clusters of fluorophores. In some embodiments, the selectively modifying comprises locally photomodifying a fluorophore labeled single DNA polymer.

In some embodiments, the data encodable nucleic acid polymer may comprise double-stranded data encodable nucleic acid polymer. In some embodiments, the data encodable nucleic acid polymer may comprise double-stranded DNA polymer. In some embodiments, the data encodable nucleic acid polymer may comprise a size of about 1 kbp to about 100 kbp. In some embodiments, the data encodable nucleic acid polymer may comprise a size of about 40 kbp.

In some embodiments, the data encodable nucleic acid polymer may comprise a plurality of fluorophores. In some embodiments, the data encodable nucleic acid polymer may comprise a plurality of fluorophores, wherein a uridine may be labeled with the fluorophore. In some embodiments, the fluorophore may be iteratively spaced along the polymer. In some embodiments, the fluorophore may occur every 1, 2, 3, 4, 5, or more bp's. In some embodiments, the fluorophore occurs every 4 bp.

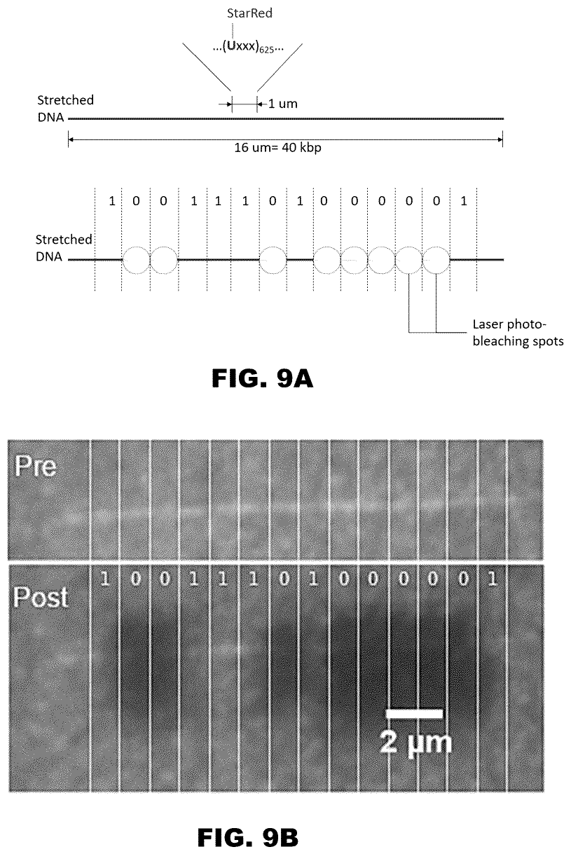

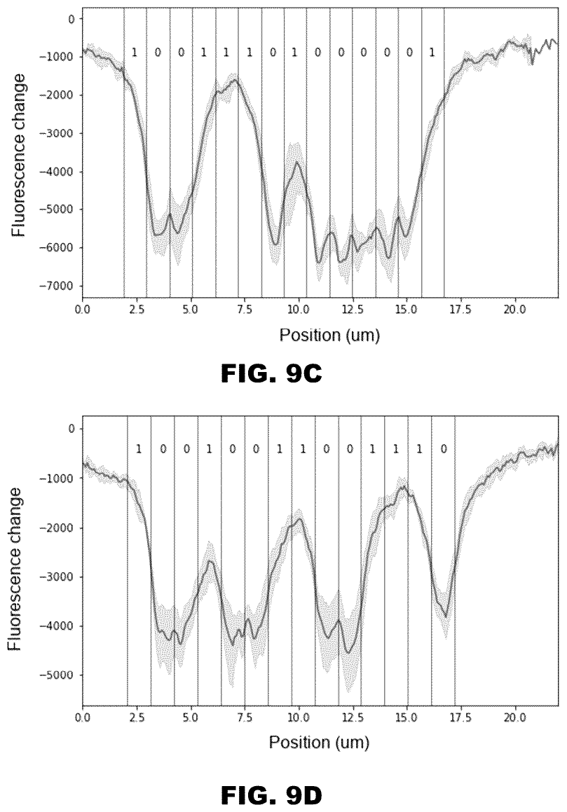

In some embodiments, the data encodable nucleic acid polymer may be labeled with about 625 StarRed fluorophores/μm (on uridine every 4 bp). In some embodiments, the 40 kbp double-stranded DNA polymer may labeled with about 625 StarRed fluorophores/μm (on uridine every 4 bp) and stretched on glass to approximately 16 μm in length as depicted in FIG. 9A. The x's in FIG. 9A indicate either adenine, cytosine or guanine residues.

In some embodiments, the selectively modifying may comprise photobleaching using a laser. In some embodiments, the selectively modifying may comprise photobleaching using a laser, wherein the photobleaching occurs at specific locations along the DNA polymer to encode data. In some embodiments, a laser may be focused to a spot (e.g., a laser spot) on the DNA polymer comprising fluorophores. In some embodiments, a laser may be focused to a spot (e.g., a laser spot) on the DNA polymer comprising fluorophores, wherein the laser is used to bleach (e.g., photobleach) fluorophores at specific locations along the polymer.

In some embodiments, as depicted in FIG. 9A, a 40 kb region of double-stranded DNA may be constructed to contain approximately 10,000 fluorescent dyes (StarRed, conjugated to deoxyuridine) repeating along every four base-pairs in the sequence.

In some embodiments, the dye may comprise a fluorescent dye. In some embodiments, the fluorescent dye may comprise an excitation maximum at about 300 nm to about 800 nm. In some embodiments, the fluorescent dye may comprise an excitation maximum at about 638 nm. In some embodiments, the fluorescent dye may comprise an emission maximum at about 300 nm to about 800 nm. In some embodiments, the fluorescent dye may comprise an emission maximum at about 655 nm. In some embodiments, the dye may comprise an excitation maximum at about 638 nm and emission maximum at about 655 nm. In some embodiments, the dye may comprise an excitation maximum at ca. 638 nm and emission maximum at ca. 655 nm. In some embodiments, the dye may comprise an excitation maximum at about 638 nm and emission maximum at about 655 nm.

In some embodiments, the DNA may be stretched on glass. In some embodiments, the DNA may be stretched on glass, wherein the stretched DNA comprises a length. In some embodiments, the stretched DNA length may be about 0.1 μm to about 1000 μm. In some embodiments, the stretched DNA length may be about 1 μm to about 100 μm. In some embodiments, the stretched DNA length may be greater than about 1000 μm. In some embodiments, the stretched DNA length may be about 16 μm.

Described herein are various methods comprising stretching DNA. In some embodiments, methods may comprise stretching DNA and immobilizing the DNA on a surface. In some embodiments, methods may comprise stretching DNA and immobilizing a surface comprising glass. In some embodiments, methods may comprise observing single DNA molecules. In some embodiments, the methods for stretching and immobilizing DNA may comprise the FiberComb® Molecular Combing System by Genomic Vision.

In some embodiments, a stretched writable DNA polymer may comprise a density of about 10 fluorophores/μm to about 1000 fluorophores/μm. In some embodiments, a stretched writable DNA polymer may comprise a density of about 500 fluorophores/μm to about 600 fluorophores/μm. In some embodiments, a stretched writable DNA polymer may comprise a density of greater than about 1000 fluorophores/μm. In some embodiments, a stretched writable DNA polymer may comprise a density of less than about 1000 fluorophores/μm. In some embodiments, a stretched writable DNA polymer may comprise a density of approximately 625 fluorophores/μm.

In some embodiments, the selectively modifying comprises use of a laser. In some embodiments. In some embodiments, the selectively modifying comprises use of a laser comprising a wavelength of about 640 nm or more. In some embodiments, the selectively modifying comprises use of a laser comprising a wavelength of 640 nm or less. In some embodiments, the selectively modifying comprises use of a laser comprising a wavelength of about 640 nm.

In some embodiments, the selectively modifying may comprise use of a laser to form photobleached spots on the stretched writable polymer. In some embodiments, the stretched writable polymer may be a DNA polymer. In some embodiments, the spots may comprise a diameter of about 1 μm. In some embodiments, the spots may comprise a diameter of greater than about 1 μm. In some embodiments, the spots may comprise a diameter of less than about 1 μm.

In some embodiments, the selectively modifying may comprise a duration of less than about 25 msec. In some embodiments, the selectively modifying may comprise a duration of greater than about 25 msec. In some embodiments, the selectively modifying may comprise a duration of about 25 msec.

In some embodiments, the selectively modifying may comprise using a laser comprising a laser power. In some embodiments, laser power may be less than about 60 mW. In some embodiments, the laser power may be greater than about 60 mW. In some embodiments, the laser power may be about 60 mW.

In some embodiments, the selectively modifying may comprise steering a laser beam, automatically from one position (e.g., photobleached spot) to the next position.

Images acquired before and after selectively modifying by photobleaching with a laser in accordance with some embodiments are depicted FIG. 9B as “pre” and “post,” respectively. An average trace of ten photobleached DNAs is shown in FIG. 9C. A second example of photobleaching DNA to encode the character “IO” (1001001 1001110) is depicted in FIG. 9D.

In certain aspects, provided herein are various methods for data encoding and data reading on polymers utilizing prefabricated clusters of fluorophores. In some embodiments, the polymer are DNA polymers. In some embodiments, the polymer may be stretched and immobilized onto a substrate. In some embodiments, the polymer may be stretched and immobilized onto a substrate and may comprise alternating sections.

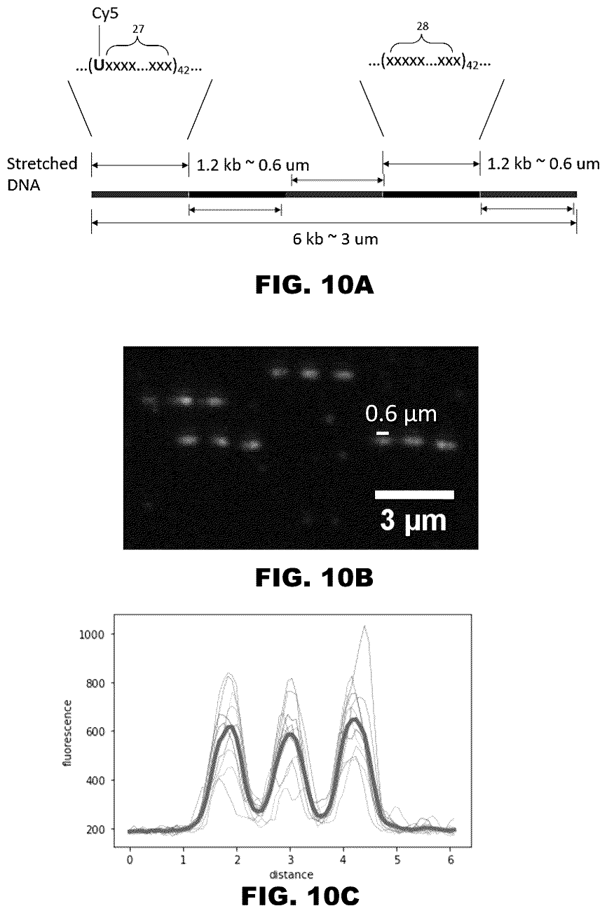

In some embodiments, as depicted in FIG. 10A the stretched DNA polymer may comprise a size of about 6 kb. In some embodiments, the size of the stretched DNA polymer may be greater than about 6 kb. In some embodiments, the size of the stretched DNA polymer may be less than about 6 kb.

In some embodiments, the alternating sections may comprise DNA residues. In some embodiments, the alternating sections may comprise fluorophores. In some embodiments, the alternating sections may comprise DNA residues and fluorophores. In some embodiments, the fluorophores may comprise a Cy5 fluorophore. In some embodiments, the DNA residues may comprise a Cy5 labeled uridine.

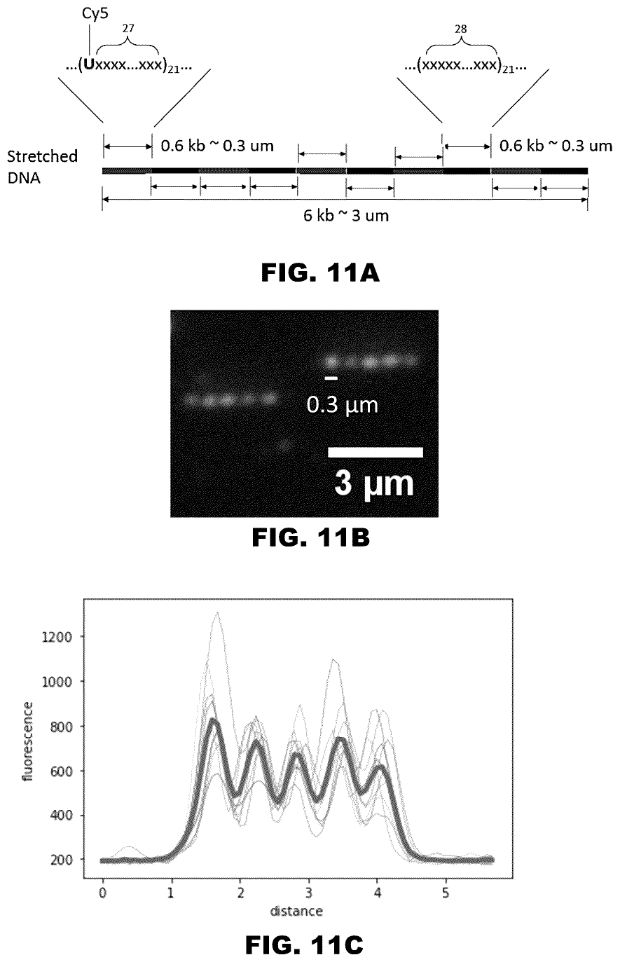

In some embodiments, the alternating section may comprise a size of about 0.1 kb or less. In some embodiments, the alternating section may comprise a size of about 2 kb or more. In some embodiments, the alternating section may comprise a size of 1.2 kb as depicted in FIG. 10A. In some embodiments, the alternating section may comprise a size of 0.6 kb as depicted in FIG. 11A.

In some embodiments, the alternating section length may comprise about 0.1 μm or less. In some embodiments the alternating section length may comprise about 1 μm or more. In some embodiments, the alternating section may comprise a length of about 0.3 μm. In some embodiments, the alternating section may comprise a length of about 0.6 μm.

In some embodiments, the alternating section may comprise 10 base pairs or less. In some embodiments, the alternating section may comprise 100 base pairs or more. In some embodiments, the alternating section may comprise 10 base pairs to 100 base pairs. In some embodiments, the alternating section may comprise 28 base pairs.

In some embodiments, the alternating section may comprise 10 fluorophores or less. In some embodiments, the alternating section may comprise 10 fluorophores or more. In some embodiments, the alternating section may comprise 10 fluorophores to 1000 fluorophores. In some embodiments, the alternating section may comprise 42 fluorophores as depicted in FIG. 10A. In some embodiments, the alternating section may comprise 21 fluorophores as depicted in FIG. 11A.

FIG. 10B depicts fluorescence images of copies of the DNA polymer described in FIG. 10A, where the DNA is stretched out on a glass substrate in accordance with some embodiments. FIG. 10C plots fluorescence intensity versus distance (m). FIG. 10C depicts a plot of 10 individual traces (e.g., fluorescence line profiles) extracted from fluorescence images of the stretched DNA polymers. The thick line of FIG. 10C represents the average of the individual traces.

FIG. 11A depicts a DNA polymer comprising alternating sections, wherein each alternating section is 0.6 kb, 0.3 μm in length and comprises 21 fluorophores, in accordance with some embodiments. FIG. 11B depicts a fluorescence image of the DNA polymer described in FIG. 11A. FIG. 11C, depicts a plot of 10 individual traces (e.g., fluorescence line profiles) extracted from fluorescence images of the stretched DNA polymers. The thick line of FIG. 11C represents the average of the individual traces.

### EXAMPLES

Described herein are various examples of compositions, systems, and methods for data storage utilizing polymers. Examples of installing chemically modifiable groups onto polymers, methods to writing data, and methods for reading data are provided.

### Example 1: Generating Data Encodable DNA

In this example, a DNA strand of repeating sequence (GTA)ₙ is constructed enzymatically by rolling circle amplification. During the polymerase extension, dGTP, dATP, and fluorescein-dUTP are provided, such that fluorescein is incorporated every third nucleotide. In a separate rolling circle reaction, single-stranded DNA with a sequence of (TAC)ₙ is prepared, by use of polymerase incorporation of rhodamine-dUTP. Once polymer strands of desired lengths are generated, the two polymer strands are isolated away from polymerase and unreacted dNTPs. The strands are then hybridized in an ionic strength buffer that supports hybridization. The product is many long DNA duplexes containing the two dyes near one another on opposite strands, similar to the example portrayed in FIG. 3B.

### Example 2: Data Encoding Via Photobleaching Clusters of Fluorophores

In this example, a modified DNA molecule is constructed to contain a sidechain at thymidine residues, where the sidechain carries photobleachable fluorophores fluorescein (green) and rhodamine (red) dyes linked to an amine group on thymine bases. Encoding is performed by passing the DNA through a plasmonic nanopore having optical resolution of 5 nm. As the DNA is passed through the pore, a pulse of light at a wavelength to excite fluorescein is impinged on the plasmonic structure, resulting in focusing high energy on a contiguous string of five of these bit modules. This results in bleaching of fluorescein in a cluster of five, yielding a written “zero” bit. As the DNA passes further, it leaves a small segment of unbleached dyes between. As the DNA passes further, a pulse of light to excite rhodamine is impinged on the device, resulting in excitation and photobleaching of a cluster of five rhodamine dyes. This represents a “one” bit written as a cluster of dyes. Reading this DNA involves observing a red-predominant signal, a mixed green/red signal, and a predominantly green signal. This signifies a code (left to right) of a zero, a space, and a one.

### Example 3: Preparation and Localized Photobleaching of Cy-5-Labelled DNA

This example demonstrates stretching and writing a synthetic DNA with fluorophores (Cy-5) labelled.

In this example, the following sequence (SEQ ID NO: 1) was used:

ATTCGCGGCCGCAGGCTAGGTGGAGGCTCAGTGATGATAAGTCTGCGAT
GGTGGATGCATGTGTCATGGTCATAGCTGTTTCCTGTGTGAAATTGTTA
TCCGCTCAGAGGGCACAATCCTATTCCGCGCTATCCGACAATCTCCAAG
ACATTAGGTGGAGTTCAGTTCGGCGTATGGCATATGTCGCTGGAAAGAA
CATGTGAGCAAAAGGCCAGCAAAAGGCCAGGAACCGTAAAAAGGCCGCG
TTGCTGGCGTTTTTCCATAGGCTCCGCCCCCCTGACGAGCATCACAAAA
ATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACC
CTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGT
TCGCTCCAAGCTGGGCTGTGTGCACGAACCCCCCGTTCAGCCCGACCGC
TGCGCCTTATCCGGTAACTATCGTCTTGAGTCCAACCCGGTAAGACACG
ACTTATCGCCACTGGCAGCAGCCACTGGTAACAGGATTAGCAGAGCGAG
GTATGTAGGCGGTGCTACAGAGTTCTTGAAGTGGTGGCCTAACTACGGC
TACACTAGAAGAACAGTATTTGGTATCTGCGCTCTGCTGAAGCCAGTTA
CCTTCGGAAAAAGAGTTGGTAGCTCTTGATCCGGCAAACAAACCACCGC
TGGTAGCGGTGGTTTTTTTGTTTGCAAGCAGCAGATTACGCGCAGAAAA
AAAGGATCTCAAGAAGATCCTTTGATCTTTTCTACGGGGTCTGACGCTC
TATTCAACAAAGCCGCCGTCCCGTCAAGTCAGCGTAAATGGGTAGGGGG
CTTCAAATCGTCCGCTCTGCCAGTGTTACAACCAATTAACAAATTCTGA
TTAGAAAAACTCATCGAGCATCAAATGAAACTGCAATTTATTCATATCA
GGATTATCAATACCATATTTTTGAAAAAGCCGTTTCTGTAATGAAGGAG
AAAACTCACCGAGGCAGTTCCATAGGATGGCAAGATCCTGGTATCGGTC
TGCGATTCCGACTCGTCCAACATCAATACAACCTATTAATTTCCCCTCG
TCAAAAATAAGGTTATCAAGTGAGAAATCACCATGAGTGACGACTGAAT
CCGGTGAGAATGGCAAAAGCTTATGCATTTCTTTCCAGACTTGTTCAAC
AGGCCAGCCATTACGCTCGTCATCAAAATCACTCGCATCAACCAAACCG
TTATTCATTCGTGATTGCGCCTGAGCGAGACGAAATACGCGATCGCTGT
TAAAAGGACAATTACAAACAGGAATCGAATGCAACCGGCGCAGGAACAC
TGCCAGCGCATCAACAATATTTTCACCTGAATCAGGATATTCTTCTAAT
ACCTGGAATGCTGTTTTCCCGGGGATCGCAGTGGTGAGTAACCATGCAT
CATCAGGAGTACGGATAAAATGCTTGATGGTCGGAAGAGGCATAAATTC
CGTCAGCCAGTTTAGTCTGACCATCTCATCTGTAACATCATTGGCAACG
CTACCTTTGCCATGTTTCAGAAACAACTCTGGCGCATCGGGCTTCCCAT
ACAATCGATAGATTGTCGCACCTGATTGCCCGACATTATCGCGAGCCCA
TTTATACCCATATAAATCAGCATCCATGTTGGAATTTAATCGCGGCCTC
GAGCAAGACGTTTCCCGTTGAATATGGCTCATAACACCCCTTGTATTAC
TGTTTATGTAAGCAGACAGTTTTATTGTTCATGATGATATATTTTTATC
TTGTGCAATGTAACATCAGAGATTTTGAGACACAACGTGGCTTTCCCCC
GCCGCTCTAGAACTAGTGGATCCAAATAAAACGAAAGGCTCAGTCGAAA
GACTGGGCCTTTCGTTTTATCTGTTGTTTGTCGCATTATACGAGACGTC
CAGGTTGGGATACCTGAAACAAAACCCATCGTACGGCCAAGGAAGTCTC
CAATAACTGTGATCCACCACAAGCGCCAGGGTTTTCCCAGTCACGACGT
TGTAAAACGACGGCCAGTCATGCATAATCCGCACGCATCTGGAATAAGG
AAGTGCCATTCCGCCTGACCTTGCCTCTCGCCAGCATTGTAATAGGCCT
CAGCTACCTGGGTACCTGAATCCATCGCTGCGTAGCTGGATAGGTGCGT
GACTAAATGAGTAAGTGAGTCGCTACATCCCTACATCCGTCGCTGGCTA
ACTCACTGCGTAACTCCATGGCTGAATGAGTCGATAGCTCGCTGAATGA
GTACGTGAATCAGTAAGTACGTAGATCGGTGGCTGCGTACCTGAATCCC
TCAATGAATCACTAGGTCACTAACTCAATACATGCCTAGGTAGGTAAGT
AAGTCAATGACTGGCTGGCTGAATCGATAACTGCATAGCTACATCAGTA
GATACATAAGTGAATGAGTCCGTAGATGGGTAACTCCATGAATCACTGC
CTGAATCAGTCAATCACTACGTAGGTAACTAGATAAATCGGTGAATACG
TGCGTGAATGGATAGGTAGATAGGTAGCTGGATCCGTCCCTAACTCGCT
CAGTGCGTAGATCACTGAATCAGTCGATCCGTACGTACCTCAGTCGATA
CGTGAGTAGCTAGGTCGCTCGCTCAATGGGTAAATACATACATCACTGC
CTGCGTGAGTCCGTGCATCGATAAGTGGATGGCTAAGTGGCTGAGTAAC
TCAATGAATACATACGTGAGTCGCTGCCTAGATGCATCGCTACCTGAAT
GAATACCTGCGTGGGTCAGTGCCTAGCTGGGTCAGTAACTCGATGAGTC
CCTAGATCCGTCACTCAGTAGCTGAATGCATCGCTCGCTAGGTAACTCC
CTCGATACATCCATAGATACGTCACTGGCTGGATCACTCGATGAATGCA
TCACTCAGTCAATCCATCGCTCACTCGCTCCCTGGGTGACTCAATGCGT
CAGTGAATAAGTGACTCGCTGGATGGATGCATCAGTCAGTGCATCACTA
AATGGGTAGATACGTACCTACCTACATCAGTCCATCACTCACTACCTAA
ATGAGTGGCTGGATCGGTCGGTCACTAAGTACGTACCTCCCTAACTCAC
TCGGTGCCTAACTCGATAGGTACCTAAATCAGTGAATACGTGCCTCGGT
AGCTAGATAAGTGCCTCGATGAATACGTAACTAGATACATCCGTGGGTG
GCTCCGTGGATCGGTGAGTGGGTCGATGCCTCACTGCCTGAGTAAATGC
CTCACTGGCTGCCTACGTGACTACATCCCTGCGTGGCTCACTCACTCGG
TGCGTAGATCAGTCCCTGGCTGCGTCCCTGCGTGCCTGGCTGAATAGGT
GGATGCGTACATCAGTACATCCCTGCATGGCTCGGTGCATACCTGACTC
CGTCACTGGCTCAGTACCTCGATAAGTGACTCGGTCAATCAGTGCGTGG
GTCCATCAATCAATCACTGGCTCCCTCAGTCCATCAATACATGGCTGAG
TGAATACATACCTGGATGCATCACTGGGTGGCTGGCTGGATACCTAAGT
ACGTAGGTGCCTACATAAGTAACTAAGTCGCTGCATCGATCCGTACCTG
CGTCAATGGCTAGGTCCGTCCGTAAGTCGATACGTGAGTCCATGGATGC
ATACATCGATACGTCCATAGATCGATACGTGCATACATAAGTCGCTACG
TGAGTAACTACCTACATGAATACATCCATCCATGCGTGACTAAGTAGAT
CCGTGGCTGGATCACTACATCGATCGCTGCGTGCGTGCGTCACTAGCTC
AGTGGATACGTGAATGAATGAATACATCGGTCACTCGCTAGGTGCATAA
GTCGATGAATAGGTGCATACGTGAGTAACTAAATGCCTCGCTGGATGAA
TCACTGGCTGAATAGATAACTGCGTAGGTGGGTAGGTAGCTCCATCGGT
GGCTCCATGGGTGAATCCCTAACTGGATGGGTGCCTACCTAACTGAGTA
GCTGGGTCGATAAATCGATCCCTCCGTAACTAACTACATCGCTGCGTAC
CTGCGTGGATCGCTGAATCGGTCCCTGAATCGATCACTAGATAAATACC
TCGATCGCTAAATGGCTGCCTGGATAGGTGAATAGCTAGGTGGGTACCT
CACTAGATGCATAGCTCCATGACTCCCTCCATCGATACGTCGGTGACTC
CCTGGCTAGGTGCCTAGGTGACTCCATACGTGCGTAGCTGGATGACTCG
GTGCGTCCATCGGTGGGTGGCTAAGTCGCTACATCCGTGGGTGCCTCGA
TCCATCCCTGGATCCATAAGTCCATACGTGCATACATGACTACCTAGCT
CGGTGGATACATCGCTCCATGACTGCATCAGTACGTAAATAACTCGGTA
GATGCATAGATACATGGCTGCGTGACTCGATCAATGAATAGGTCAATAG
CTCCGTCGCTGCCTCGCTAGCTGGGTGGATGACTACGTGGATACATAAG
TGGATGAATGGCTGGCTGGATCCCTCCCTCCATCACTGGCTACCTAAGT
GAATAACTACCTAACTAGCTAGATGGCTGGGTCCATCAATGGCTAGATC
CCTCAATAGGTACATCGGTCCATGCATCCATAACTACGTCAGTCCATAC
GTCCATCCATGAGTCGATCGATGGGTAGGTACATACGTAAATGCGTAAG
TCAATCCCTACATGCCTACATCACTAGGTGGATGAGTCGGTGGATGCGT
AGCTGCATCCCTAGGTACATCACTCCGTGAGTAAATACCTACATGGCTA
GGTAAATCCATAAGTCGGTCACTCGCTCCGTCCCTGCGTCCCTGCCTAA
GTAGATGAATAGGTCACTGCATGCCTAGCTAACTAGATCCCTAAGTACA
TGAGTAAATCAATCCCTGCATGCCTCGGTGCATACATAGGTGAGTGAAT
GAGTCGCTACGTAACTCACTCAATCGATAAGTCAGTACCTCCCTGCCTA
CCTGACTAGGTGCGTAAATAGATAAATACGTGGCTCCATACATAAGTAG
GTGGCTAACTCACTAGCTGGATGGCTCAATCGGTGCCTCGCTAGCTCGC
TGCGTAACTCGGTAAATCACTAGCTAACTGCGTCCCTGAGTAGCTCCGT
AACTAGCTAAGTGCCTAACTCAGTGCGTAGCTAACTAACTAGCTCGCTG
AGTCACTGCATCAATCAATGCATAACTGGGTGAATAAATCAATACCTCC
ATGAGTAGATAAATAAGTGCGTCGGTGAATGCCTGAGTGACTCGATCAG
TCAGTGCGTGGCTAGGTGGGTCGGTCCGTCACTGGCTCGCTACGTGGGT
GACTGCCTGCCTCCGTACGTACCTGCGTAGGTACATAGATACCTCAGTA
ACTCGCTGCCTCGCTAGCTGGGTACGTGGATAACTAGATCGGTAGGTCC
ATGAATCAATACGTACATCCCTCAATACGTGAGTAAATCCCTCACTCGC
TAAATGGCTGACTACGTCCGTCCGTGACTAAGTAGGTAAGTCGCTGGAT
AACTCAATACCTGGCTACATAGCTCAGTCGGTACCTAAATGCCTCAATG
GCTCGCTGAGTCGGTCAATCCCTCCATCCGTAACTCACTAAGTCAGTCC
ATGCCTAGGTAACTCCCTCGATAACTAACTGGGTAAATGACTAACTCGC
TACGTAAATGCCTGCGTACGTCAATAGATCACTCCCTGAATGGGTGGCT
GGCTGGGTCGCTAGCTCCATCCATACATGGCTCCGTGCGTCGCTACATC
AATGCGTAGCTCGGTCAATGCCTGCGTACGTCGATAAATAGCTGCCTAC
GTGAGTAGATGGGTCCCTCAATCCCTGAATAAGTACGTAAGTCCGTACC
TCAATACCTAACTGCCTGCATCAATACCTAGATAAGTGCGTGACTGAAT
AGGTCAGTCGATCGCTAGGTCAATGAGTGGCTCGATGCATGGGTAGATA
GATGCGTGGATAGATACGTGCCTGGCTGAATAGGTAGATGCCTACATCC
ATGACTCGCTCGGTCGGTCACTCAATGGATAAATGACTAAGTACGTCGC
TGCATCACTCCATCCGTCCCTAAGTAGGTGGCTACGTCAGTCGATGAGT
GGGTGAGTAGATCGATAACTGAATAGCTCAATAAATGGGTCCATGGCTG
GCTGGATGCGTCGGTGAGTGCATGGGTCCGTACATCGGTCACTCCCTCG
ATGGCTCCATCGGTCCATGCCTGGCTCGATACGTGAATAGATACATAGC
TGACTGGGTCACTAAGTGAGTGCGTCCATGCATCCCTACGTACGTGAGT
CAGTACGTCAATGCGTAACTACGTGCATGAATAACTCGATCCGTCAGTG
CCTAAGTGAATAGCTGGCTAACTGGATCACTGCATAGCTCCATCCCTGG
CTCCATGGATGAATCGCTGCATCCATAACTCGATCCGTACGTACGTCAA
TAGGTGACTCCCTGAGTCAGTAGATGGCTACGTCGATGCCTAGATAGGT
GGATGCGTACCTCGATAGGTAACTACGTAAGTCGATCGATCCATCCGTG
CCTGCGTGGATCGGTAGATAACTAGCTACATCCCTCCATGCATACATAC
ATAAATGAATGACTCGCTCCATCACTGCGTGACTGCATACGTGAATAAA
TACGTGCGTGCCTCGATACATGCGTGACTCAATGACTCGCTCCGTCCGT
GCGTCCGTAACTCGGTAAATCGGTACATGCCTGCATACATCAGTGCCTC
CGTCCCTGCATACGTCCGTCCATCGATCGATAGCTGGCTAAATAGGTGG
GTAGGTAGATGCCTGCGTCGATAGATGAATACATCGGTAAATGGCTCGC
TACGTAAGTAGGTGGGTAGATCAGTGGGTACGTCGCTAGCTAGATAGGT
CGATACATAACTGACTCACTCGGTCCGTACGTGGGTAAATCAATAGCTA
GCTACCTGCGTACCTGCATCGATGGCTAAATCCATACGTCCATAGATCA
CTCCCTGCATCGGTGAATGGGTGAATCAATCAGTGCGTCCCTGGGTACG
TGGCTAAATAACTAGATGGCTGCATAGGTAACTGCATCGATACATCACT
ACGTACATCCATAAGTCCGTCCATAAGTCGATCGCTAGATAAATGAGTG
CGTAAATGAGTGCCTCGGTGGATAGATGCGTACCTCGATAGGTGCGTGG
CTACCTCGGTACGTCCGTCCCTAGATGGCTAGGTGCATAACTGGCTACG
TAACTCGCTCCGTCGGTGCCTACCTAAATAAGTCCGTGCGTCCCTGCAT
CCATCCCTAAATCGATAAGTCGCTAGCTCGATCAGTAAGTACATCCCTG
AATCAATAAGTAAATCCCTCCGTACGTGAGTGAGTGCGTAGGTGCATCA
GTGGATAAGTGGGTGCATCACTGCGTCCATGCATCACTACCTAAGTCCG
TGCGTAGGTAGATCAGTACGTGAGTGCCTAAGTGCGTAGCTACGTAAGT
GAGTGACTGAGTGCCTGGGTCCATGAATACCTCCATACGTGGGTACCTA
CGTCGGTGACTAGGTCACTGCATACCTGGGTCCGTAGGTACCTAGCTGC
ATAGATAAGTCCCTGCGTGGATGCCTCCGTGGCTGAATCGGTCAATGCA
TGGATAAATACGTGCATCGCTGAATGGCTCAGTCAATACATCAGTGG.

### DNA Preparation Using Plasmid

DNA with the above sequence (SEQ ID NO: 1) was prepared using a plasmid and a DNA polymerase by replacing T's with dye-labelled dUTPs (such as Cy5-dUTP), affording DNAs with labeled U's, e.g. (where N may be A or C or G):

......NNNNNNNNNUNNNUNNNUNNNUNNN......

### DNA Clean Up

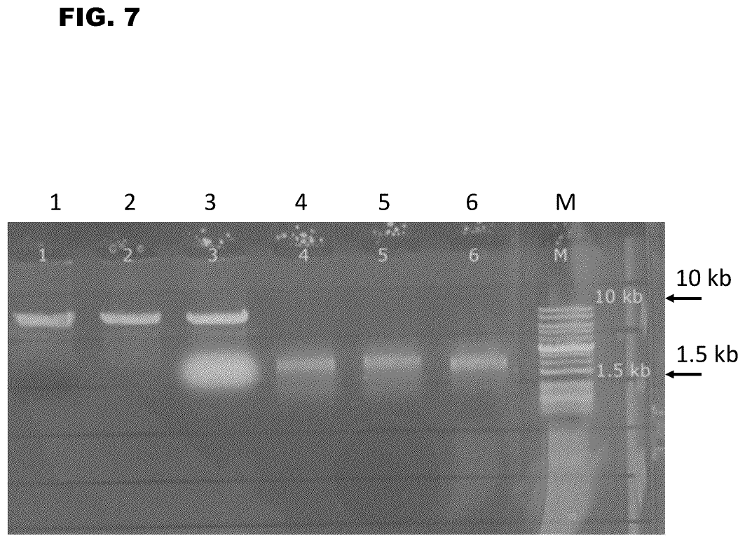

The reaction mixture containing the prepared DNA was mixed with 100 μL of CleanNGS DNA & RNA Clean-Up Magnetic Beads, incubated at room temperature for 5 minutes, and left on magnetic stand for 3 minutes. The clear liquids were aspirated and discarded. The magnetic beads were washed with 400 μL of 70% Ethanol 2 times with one minute incubation each time. After ethanol wash, the beads were left dried for one minute and then incubated with 50 uL IDTE buffer for 5 minutes on a rack. The tube was then moved back to the magnetic stand and left for 3 minutes. After all the magnetic beads moved to the side, the clear liquid was transferred to a new 1.5 mL Eppendorf tube. The concentration of DNA was measured using Qubit fluorometer and the integrity was checked using gel electrophoresis. Typically, 500-1000 ng labeled DNA was recovered. An example of labeled DNA electrophoresis can be seen in FIG. 7, where Lane 1 contains Cy5-labeled DNA and Lane M contains Safegreen 1 kb DNA ladder.

### DNA Combing and Stretching

Materials for the DNA combing protocol included: The Fibercomb instrument, genomic DNA extraction kit, stretching reservoirs and vinylsilane coated coverslips purchased from Genome Vision. Prolong Gold mounting solution was from Thermofisher, and YOYO-1 was from Biotium.

In the DNA stretching protocol, 1 μL of labeled DNA (20-50 ng) was diluted in 2.4 mL of Buffer 6 from the genomic DNA extraction kit. The solution was warmed to 37° C. for 5 minutes and then loaded into the reservoir. The vinyl-silane coated coverslips were incubated in the reservoir for 3 minutes and pulled up slowly, causing DNA to stretch on the coverslip using the Fibercomb instrument. The coverslips were heated to 125° C. for 3 minutes and then mounted on a slide with 10 μL of Prolong Gold mounting solution with 1 uM YOYO-1. The coverslips were sealed with nail polish.

### DNA Bleaching and Imaging

Stretched DNA on coverslips were imaged using a Cytiva OMX-SR microscope. The slide with stretched DNA was loaded on the microscope per manufacturer's instructions. A rectangle box perpendicular to the stretched DNA was used to bleach segments of the Cy5 labeled DNA for 25 milliseconds using a 633 nm laser.

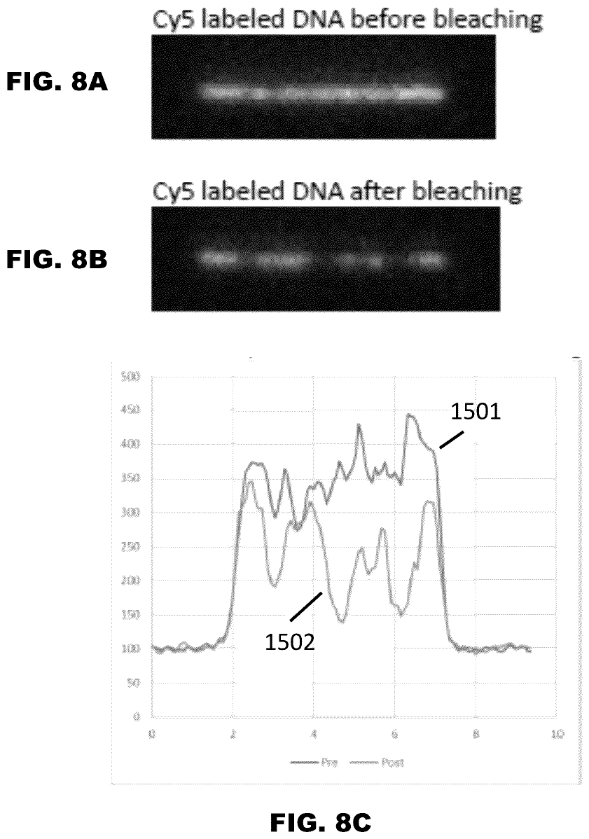

The prepared DNA with Cy-5 labelled Us was locally photobleached with light to write on the DNA. Results for DNA imaging and bleaching are shown in FIGS. 8A-8C. Y axis shows emission intensity, and X axis is distance (microns).

FIG. 8A shows an image of Cy5 labeled DNA before bleaching and FIG. 8B shows an image of Cy5 labeled DNA after sequential photobleaching. FIG. 8C shows a plot displaying two cross section profiles: before bleaching (1501) and after bleaching (1502), showing three distinct sites of bleaching, observable in darkened spots in the DNA and as valleys in the intensity plot. The peak to valley distance is about 700 nm. From FIG. 8C the separation of the pre-bleached state represented by the pre-bleach profile (1501) and post bleached state represented by the post-bleach profile (1502) can be clearly seen.

### Example 4: Data Encoding Via Photobleaching Clusters of Fluorophores

In this example, as depicted in FIG. 9A, a 40 kb region of double-stranded DNA was constructed to contain approximately 10,000 fluorescent dyes (StarRed, conjugated to deoxyuridine) repeating along every four base-pairs in the sequence. StarRed dye has an excitation maximum at ca. 638 nm and emission maximum at ca. 655 nm. The DNA was then stretched on glass to a length of ˜16 μm using the FiberComb Molecular Combing System by Genomic Vision which allows single DNA molecules to be observed. A first image was taken as shown in FIG. 9B (labeled as “pre”). This stretched writable DNA polymer corresponded to a density of approximately 625 fluorophores/μm and was monitored under a Cytiva OMX-SR microscope. A 640 nm laser was focused and used to photobleach spots of about 1 μm in diameter on the DNA, yielding several “zero” bits, to encode the characters “NA” in binary (10011101000001). The bleaching was carried out for 25 msec at each spot using 60 mW laser power. The laser beam was steered automatically from one position to the next position. After bleaching, a second image was taken to read the written information, as is shown in FIG. 9B (labeled as “post”). Ten traces (e.g., fluorescence line profiles) of photobleached DNAs are shown in FIG. 9C. A second example of photobleaching DNA was also performed to encode the character “IO” (1001001 1001110) as shown in FIG. 9D. In this way, approximately 625 dyes as a cluster were used to encode each binary “bit” of information, and consecutive bits encoded a string of digital information. Note that traces of fluorescence quantified along the DNA were read individually for each DNA polymer. Each trace was averaged for better accuracy in analyzing the string of encoded bits as shown in FIGS. 9C and 9D.

### Example 5: Data Encoding and Reading Via Prefabricated Clusters of Fluorophores

In this example, a 6 kb region of double-stranded DNA was constructed to contain alternating 1.2 kb sections. Each alternating section contained either Cy5-linked deoxyuridines every 28 base-pairs (to a total of 42 fluorophores/section), or only deoxyadenine, deoxycytosine, and deoxyguanine residues, as depicted in FIG. 10A. The Cy5-deoxyuridine containing sections were fluorescent, yielding “one” bits, while the Cy5-uridine absence sections remained dark, yielding “zero” bits. To read this digital information in the polymer, the DNA was then stretched using the FiberComb Molecular Combing System by Genomic Vision on glass to a length of about 3 μm and monitored under a Cytiva OMX-SR microscope. Upon imaging, alternating bright and dark stripes, where each stripe was measured to be 0.6 μm in length, were observed as depicted in FIG. 10B. A plot of 10 traces (e.g., fluorescence intensity line profiles) of separate stretched DNA segments (thin lines) and the average (thick line) is depicted in FIG. 10C. The plot depicts the binary string “10101”, confirming the ability to accurately read bits in single DNAs optically. In this example, clusters of 42 dyes to encode “1” bits were used and similar intervals with little or no fluorescence to encode “0” were used.

Similarly, a second DNA was constructed with alternating sections containing 0.6 kb and Cy5. In this example, the sections alternated between sections comprising 21 fluorophores and sections comprising no fluorophores as depicted in FIG. 11A. In this case, bright and dark stripes, where each stripe was measured to be about 0.3 μm in length, were observed as shown in FIG. 11B. A plot of 10 traces of separate stretched DNA segments (e.g., thin lines) and the average (e.g., thick line) is depicted in FIG. 11C, confirming the encoding of the binary string “101010101.” This data confirms that clusters of 21 fluorophores in a DNA polymer can be used to encode single bits, and that the digital string can be read on a single molecule level.

1-267. (canceled)

268. A method, comprising
(a) providing a polymer, wherein:
(i) said polymer comprises at least 100 residues;
(ii) said polymer comprises a plurality of clusters comprising a plurality of convertible residues, wherein each convertible residue of said plurality of convertible residues is in a first state, and is capable of being converted into a second state by a stimulus; and
(iii) each cluster of said plurality of residue clusters comprises at least 10 convertible residues; and
(b) encoding data into said polymer by converting convertible residues of a cluster of said plurality of clusters from said first state into said second state using said stimulus, wherein a data unit of said data is represented by said cluster.

269. The method of claim 268, wherein said plurality of convertible residues are covalently linked to said polymer.

270. The method of claim 268, wherein said polymer is a nucleic acid.

271. The method of claim 268, wherein said second state comprises a naturally occurring nucleobase.

272. The method of claim 268, wherein a conversion of said first state into said second state is an irreversible conversion.

273. The method of claim 268, wherein said plurality of convertible residues comprises non-naturally occurring nucleobases.

274. The method of claim 268, wherein each of said convertible residues of said plurality of convertible residues comprises a chemically modifiable moiety.

275. The method of claim 274, wherein said chemically modifiable moiety is directly linked to one or more nucleobases.

276. The method of claim 274, wherein said chemically modifiable moiety comprises one or more photo-removable groups.

277. The method of claim 268, wherein each convertible residue of said plurality of convertible residues of said cluster is present every 2-25 residues.

278. The method of claim 268, wherein said cluster comprises at least 20 convertible residues.

279. The method of claim 278, wherein said cluster comprises at least 625 convertible residues.

280. The method of claim 268, wherein the cluster is separated from a second cluster of the plurality of clusters by a plurality of spacers, wherein said spacers of said plurality of spacers are not capable of being converted into said second state by said stimulus.

281. The method of claim 268, wherein said polymer comprises at least 1,000 residues.

282. The method of claim 268, wherein said polymer comprises at least 10,000 residues.

283. The method of claim 268, wherein (b) comprises using a nanopore to provide said stimulus.

284. The method of claim 268, wherein (b) comprises using a nanochannel to provide said stimulus.

285. The method of claim 283, wherein said nanopore comprises a plasmonic nanopore device that provides light pulses or redox energy.

286. The method of claim 285, wherein said plasmonic nanopore device provides localized pulses of light energy and wherein said light pulses comprise a wavelength from 300-850 nm.

287. The method of claim 268, wherein said stimulus comprises light pulses.

288. The method of claim 268, wherein said stimulus comprises a redox agent.

289. The method of claim 268, further comprising after (b) reading said data encoded into said polymer.

290. The method of claim 289, wherein said reading comprises using a nanopore.

291. The method of claim 268, further comprising after (b) storing said polymer without a stabilizer.


SEQUENCE LISTING (1 sequence)

SEQ ID NO: 1 - DNA, 7740 bp, synthetic construct
  source 1..7740: mol_type = other DNA
attcgcggccgcaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttgcctctcgccagcattgtaataggcctcagctacctgggtacctgaatccatcgctgcgtagctggataggtgcgtgactaaatgagtaagtgagtcgctacatccctacatccgtcgctggctaactcactgcgtaactccatggctgaatgagtcgatagctcgctgaatgagtacgtgaatcagtaagtacgtagatcggtggctgcgtacctgaatccctcaatgaatcactaggtcactaactcaatacatgcctaggtaggtaagtaagtcaatgactggctggctgaatcgataactgcatagctacatcagtagatacataagtgaatgagtccgtagatgggtaactccatgaatcactgcctgaatcagtcaatcactacgtaggtaactagataaatcggtgaatacgtgcgtgaatggataggtagataggtagctggatccgtccctaactcgctcagtgcgtagatcactgaatcagtcgatccgtacgtacctcagtcgatacgtgagtagctaggtcgctcgctcaatgggtaaatacatacatcactgcctgcgtgagtccgtgcatcgataagtggatggctaagtggctgagtaactcaatgaatacatacgtgagtcgctgcctagatgcatcgctacctgaatgaatacctgcgtgggtcagtgcctagctgggtcagtaactcgatgagtccctagatccgtcactcagtagctgaatgcatcgctcgctaggtaactccctcgatacatccatagatacgtcactggctggatcactcgatgaatgcatcactcagtcaatccatcgctcactcgctccctgggtgactcaatgcgtcagtgaataagtgactcgctggatggatgcatcagtcagtgcatcactaaatgggtagatacgtacctacctacatcagtccatcactcactacctaaatgagtggctggatcggtcggtcactaagtacgtacctccctaactcactcggtgcctaactcgataggtacctaaatcagtgaatacgtgcctcggtagctagataagtgcctcgatgaatacgtaactagatacatccgtgggtggctccgtggatcggtgagtgggtcgatgcctcactgcctgagtaaatgcctcactggctgcctacgtgactacatccctgcgtggctcactcactcggtgcgtagatcagtccctggctgcgtccctgcgtgcctggctgaataggtggatgcgtacatcagtacatccctgcatggctcggtgcatacctgactccgtcactggctcagtacctcgataagtgactcggtcaatcagtgcgtgggtccatcaatcaatcactggctccctcagtccatcaatacatggctgagtgaatacatacctggatgcatcactgggtggctggctggatacctaagtacgtaggtgcctacataagtaactaagtcgctgcatcgatccgtacctgcgtcaatggctaggtccgtccgtaagtcgatacgtgagtccatggatgcatacatcgatacgtccatagatcgatacgtgcatacataagtcgctacgtgagtaactacctacatgaatacatccatccatgcgtgactaagtagatccgtggctggatcactacatcgatcgctgcgtgcgtgcgtcactagctcagtggatacgtgaatgaatgaatacatcggtcactcgctaggtgcataagtcgatgaataggtgcatacgtgagtaactaaatgcctcgctggatgaatcactggctgaatagataactgcgtaggtgggtaggtagctccatcggtggctccatgggtgaatccctaactggatgggtgcctacctaactgagtagctgggtcgataaatcgatccctccgtaactaactacatcgctgcgtacctgcgtggatcgctgaatcggtccctgaatcgatcactagataaatacctcgatcgctaaatggctgcctggataggtgaatagctaggtgggtacctcactagatgcatagctccatgactccctccatcgatacgtcggtgactccctggctaggtgcctaggtgactccatacgtgcgtagctggatgactcggtgcgtccatcggtgggtggctaagtcgctacatccgtgggtgcctcgatccatccctggatccataagtccatacgtgcatacatgactacctagctcggtggatacatcgctccatgactgcatcagtacgtaaataactcggtagatgcatagatacatggctgcgtgactcgatcaatgaataggtcaatagctccgtcgctgcctcgctagctgggtggatgactacgtggatacataagtggatgaatggctggctggatccctccctccatcactggctacctaagtgaataactacctaactagctagatggctgggtccatcaatggctagatccctcaataggtacatcggtccatgcatccataactacgtcagtccatacgtccatccatgagtcgatcgatgggtaggtacatacgtaaatgcgtaagtcaatccctacatgcctacatcactaggtggatgagtcggtggatgcgtagctgcatccctaggtacatcactccgtgagtaaatacctacatggctaggtaaatccataagtcggtcactcgctccgtccctgcgtccctgcctaagtagatgaataggtcactgcatgcctagctaactagatccctaagtacatgagtaaatcaatccctgcatgcctcggtgcatacataggtgagtgaatgagtcgctacgtaactcactcaatcgataagtcagtacctccctgcctacctgactaggtgcgtaaatagataaatacgtggctccatacataagtaggtggctaactcactagctggatggctcaatcggtgcctcgctagctcgctgcgtaactcggtaaatcactagctaactgcgtccctgagtagctccgtaactagctaagtgcctaactcagtgcgtagctaactaactagctcgctgagtcactgcatcaatcaatgcataactgggtgaataaatcaatacctccatgagtagataaataagtgcgtcggtgaatgcctgagtgactcgatcagtcagtgcgtggctaggtgggtcggtccgtcactggctcgctacgtgggtgactgcctgcctccgtacgtacctgcgtaggtacatagatacctcagtaactcgctgcctcgctagctgggtacgtggataactagatcggtaggtccatgaatcaatacgtacatccctcaatacgtgagtaaatccctcactcgctaaatggctgactacgtccgtccgtgactaagtaggtaagtcgctggataactcaatacctggctacatagctcagtcggtacctaaatgcctcaatggctcgctgagtcggtcaatccctccatccgtaactcactaagtcagtccatgcctaggtaactccctcgataactaactgggtaaatgactaactcgctacgtaaatgcctgcgtacgtcaatagatcactccctgaatgggtggctggctgggtcgctagctccatccatacatggctccgtgcgtcgctacatcaatgcgtagctcggtcaatgcctgcgtacgtcgataaatagctgcctacgtgagtagatgggtccctcaatccctgaataagtacgtaagtccgtacctcaatacctaactgcctgcatcaatacctagataagtgcgtgactgaataggtcagtcgatcgctaggtcaatgagtggctcgatgcatgggtagatagatgcgtggatagatacgtgcctggctgaataggtagatgcctacatccatgactcgctcggtcggtcactcaatggataaatgactaagtacgtcgctgcatcactccatccgtccctaagtaggtggctacgtcagtcgatgagtgggtgagtagatcgataactgaatagctcaataaatgggtccatggctggctggatgcgtcggtgagtgcatgggtccgtacatcggtcactccctcgatggctccatcggtccatgcctggctcgatacgtgaatagatacatagctgactgggtcactaagtgagtgcgtccatgcatccctacgtacgtgagtcagtacgtcaatgcgtaactacgtgcatgaataactcgatccgtcagtgcctaagtgaatagctggctaactggatcactgcatagctccatccctggctccatggatgaatcgctgcatccataactcgatccgtacgtacgtcaataggtgactccctgagtcagtagatggctacgtcgatgcctagataggtggatgcgtacctcgataggtaactacgtaagtcgatcgatccatccgtgcctgcgtggatcggtagataactagctacatccctccatgcatacatacataaatgaatgactcgctccatcactgcgtgactgcatacgtgaataaatacgtgcgtgcctcgatacatgcgtgactcaatgactcgctccgtccgtgcgtccgtaactcggtaaatcggtacatgcctgcatacatcagtgcctccgtccctgcatacgtccgtccatcgatcgatagctggctaaataggtgggtaggtagatgcctgcgtcgatagatgaatacatcggtaaatggctcgctacgtaagtaggtgggtagatcagtgggtacgtcgctagctagataggtcgatacataactgactcactcggtccgtacgtgggtaaatcaatagctagctacctgcgtacctgcatcgatggctaaatccatacgtccatagatcactccctgcatcggtgaatgggtgaatcaatcagtgcgtccctgggtacgtggctaaataactagatggctgcataggtaactgcatcgatacatcactacgtacatccataagtccgtccataagtcgatcgctagataaatgagtgcgtaaatgagtgcctcggtggatagatgcgtacctcgataggtgcgtggctacctcggtacgtccgtccctagatggctaggtgcataactggctacgtaactcgctccgtcggtgcctacctaaataagtccgtgcgtccctgcatccatccctaaatcgataagtcgctagctcgatcagtaagtacatccctgaatcaataagtaaatccctccgtacgtgagtgagtgcgtaggtgcatcagtggataagtgggtgcatcactgcgtccatgcatcactacctaagtccgtgcgtaggtagatcagtacgtgagtgcctaagtgcgtagctacgtaagtgagtgactgagtgcctgggtccatgaatacctccatacgtgggtacctacgtcggtgactaggtcactgcatacctgggtccgtaggtacctagctgcatagataagtccctgcgtggatgcctccgtggctgaatcggtcaatgcatggataaatacgtgcatcgctgaatggctcagtcaatacatcagtgg